(19) United States
(12) Patent Application Publication
(10) Pub. No.: US 2004/0197329 A1
(43) Pub. Date: Oct. 7, 2004

(54) Remedies or preventives for rheumatoid arthritis

(76) Inventors: Yasunori Nakayama, Tokyo (JP); Takashi Kamimura, Tokyo (JP); Tohru Akahoshi, Kanagawa (JP); Hirobumi Kondo, Kanagawa (JP)

Correspondence Address: SUGHRUE MION, PLLC, 2100 PENNSYLVANIA AVENUE, N.W., SUITE 800, WASHINGTON, DC, 20037, US

(21) Appl. No.: 10/204,228

(22) PCT Filed: Apr. 24, 2001
(86) PCT No.: PCT/JP01/03504

(30) Foreign Application Priority Data
Oct. 13, 2000 (JP) 2000-313459

(51) Int. Cl. A61K 39/395

(52) U.S. Cl. 424/143.1; 424/145.1

(57) ABSTRACT

This invention provides a therapeutic agent for treating or preventing rheumatoid arthritis containing a LARC inhibitor or a LARC receptor inhibitor.

### FIELD OF THE INVENTION

The present invention relates to remedies or preventives for rheumatoid arthritis, and more particularly, features methods of treating or preventing by administering an agent that inhibits LARC, or LARC receptor activity.

### BACKGROUND

Rheumatoid arthritis is a systemic, chronic inflammatory disease principally characterized by polyarthritis of unknown etiology and is an autoimmune disease associated with abnormal immunity. Joints of patients suffering from rheumatoid arthritis are severely infiltrated with leukocytes such as lymphocytes, and hyperproliferation of synovial cells leads to formation of pannus in joints of rheumatoid arthritis patients. Although various mechanisms of rheumatoid arthritis have been proposed to explain the cause of the disease, including heredity, microbial infection, or involvement of various cytokines and chemokines, the precise onset mechanism has not known (Feldmann et al., Cell, 1996, 85, 307).

It has recently been discovered that expression of chemokines in the joints of rheumatoid arthritis contributes to the infiltration of leukocytes into the synovium and the synovial fluid. Several such chemokines have been identified, including interleukin-8, regulated upon activation normal T-cell expressed and secreted (RANTES), macrophage inflammatory protein-1a (MIP-1a), and monocyte chemoattractant protein-1 (MCP-1).

Some reports showed that symptoms in the animal models of rheumatoid arthritis had been improved by the blockade of interaction between these chemokines and their correspondent receptors (Barns et al., J. Clin. Invest., 1998, 101, 2910; Ogata et al., J. Pathol., 1997, 182, 106). Inhibition of these chemokines, however, does not completely improve the symptoms. It is believed that aside from these chemokines, unidentified chemokines are also involved in the abnormal immune response in the joints.

The term chemokine refers to a family of inflammation/immunity-regulatory polypeptides that have molecular weights of about 10 kDa and are produced by various types of cells. Chemokines are divided into sub-groups depending on how the first two of the four N-terminal cysteine residues are arranged. Major sub-groups are CC chemokines, in which the two N-terminal cysteine residues are adjacent to one another, and CXC chemokines, in which one amino acid is inserted between the two cysteine residues. C chemokines and CX3C chemokines are also identified (Murdoch et al., Blood, 2000, 95, 3032; Rossi et al., Annu. Rev. Immunol., 2000, 18, 217).

These chemokines bind to correspondent chemokine receptors, which are a seven-transmembrane G-protein coupled receptor. Chemokines promote chemotaxis of certain types of cells and are capable of enhancing expression of cell adhesion molecules as well as cell adhesion itself. For these reasons, chemokines are considered as a protein factor that is closely associated with adhesion and infiltration of cells such as leukocytes to affected sites such as inflamed tissues (Murdoch et al., Blood, 2000, 95, 3032; Rossi et al., Annu. Rev. Immunol., 2000, 18, 217; Campbell et al., Science, 1998, 279, 381).

In addition to macrophages, cells such as dendritic cells, B-lymphocytes, and memory T-lymphocytes have been known to infiltrate into the synovium of joints of rheumatoid arthritis. Thus, these cells are thought to play an important role in provoking abnormal immune responses in the affected joints. However, in the joints of rheumatoid arthritis, chemokines that can selectively induce infiltration of these cells have not been identified yet.

Abnormal immunity involving T-lymphocytes, macrophages, and synovial cells has long been thought to be the pathogenesis of rheumatoidarthritis. However, as a result of recent findings that rheumatoid factors, autoantibodies produced by B-lymphocytes, are found in serum of about 80% of patients suffering from rheumatoid arthritis and that the infiltratation of B-lymphocytes is found in synovium of joints of rheumatoid arthritis and these B-lymphocytes are in fact capable of producing rheumatoid factors, the significance of B-lymphocytes in establishing disease state of rheumatoid arthritis has attracted considerable attention (Reparon Schuijt et al., Arthritis. Rheum., 1998, 41, 2211; Kimetal., J. Immunol., 1999, 162, 3053; Williams et al., Immunology, 1999, 98, 123).

Some reports suggest involvement of dendritic cells in the onset of rheumatoid arthritis. It is believed that a substantial number of dendritic cells are present in the regions found T-lymphocyte infiltration of synovium of rheumatoid arthritis and these dendritic cells present antigens to the T-lymphocytes, thereby activating them. On the other hand, because only a small number of dendritic cells are found in the synovial tissue of healthy people and osteoarthritis patients, T-lymphocytes also are not activated in the synovium. These observations suggest that dendritic cells present in the synovium of rheumatoid arthritis play an important role in provoking abnormal immune responses in the affected joints (Sato Kazuto, Igaku-no-ayumi, 1997, 182, 534).

It is also known that immature dendritic cells first migrate into the synovium of rheumatoid arthritis. The infiltrated immature dendritic cells differentiate into mature dendritic cells in the synovium and then present autoantigen to activate the etiologic T-lymphocytes. Since these etiologic T-lymphocytes are responsible for the subsequent activation of macrophages and B-lymphocytes, activation of T-lymphocytes, macrophages, and B-lymphocytes can be suppressed by inhibition of dendritic cell infiltration into the synovium (Thomas et al ., Immunol. Today, 1996, 17, 559; Thomas et al., Arthritis. Rheum., 1992, 35, 1455).

It is also known that the main part of the population of immigrant T-lymphocytes found in the synovium-of rheumatoid arthritis is memory T-lymphocytes. It is thus very likely that the T-lymphocytes responsible for the onset of rheumatoid arthritis are memory T-lymphocytes (Thomas et al., Arthritis. Rheum., 1992, 35, 1455).

It is therefore contemplated that devising away to inhibit infiltration of dendritic cells, B-lymphocytes, and memory T-lymphocytes into synovium will provide a novel composition for treating rheumatoid arthritis. It is also contemplated that inhibition of migration, differentiation, or, activation of dendritic cells in synovium will make it possible to treat or prevent rheumatoid arthritis at an early stage.

Until today, it has not been clarified why these cells infiltrate into the synovial tissue of rheumatoid arthritis but not those of healthy people and osteoarthritis patients, nor has any medicament been found that specifically inhibits infiltration of these cells.

It is thus an objective of the present invention to provide a therapeutic or prophylactic agent for rheumatoid arthritis that acts by preventing infiltration, differentiation, or activation of dendritic cells, B-lymphocytes, and memory T-lymphocytes in the joints of rheumatoid arthritis.

It is another objective of the present invention to provide a therapeutic or prophylactic agent for rheumatoid arthritis that contains an inhibitor of LARC, or an inhibitor of LARC receptors as an active ingredient, and acts by preventing infiltration, differentiation, or activation of dendritic cells, B-lymphocytes, and memory T-lymphocytes in rheumatoid joints.

In the, course of their studies, the present inventors have discovered that synovial cells of patients suffering from rheumatoid arthritis produce LARC, a type of chemokine capable of inducing migration of immatured endritic cells, B-lymphocytes, and memory T-lymphocytes, that LARC, as well as its receptor CCR6, is expressed in synovium of rheumatoid arthritis, and that an inhibitor capable of inhibiting binding of LARC to its receptors can serve as a therapeutic or prophylactic agent for the disease.

### SUMMARY OF THE INVENTION

Accordingly, the present invention provides a therapeutic agent for treating or preventing rheumatoid arthritis that contains an inhibitor of LARC or LARC receptors as an active ingredient.

### BRIEF DESCRIPTION OF THE DRAWINGS

FIG. 1 is the results of RT-PCR and southern hybridization performed to study the expression of LARC mRNA in synovial cells derived from a rheumatoid arthritis patient.

FIG. 2 is the results of immunostaining to examine the expression of LARC protein in synovial cells derived from a rheumatoid arthritis patient.

FIG. 3 is a diagram showing the ability of the anti-LARC antibody to inhibit migration of lymphocytes induced by the culture supernatant of the synovial cells derived from a rheumatoid arthritis patient.

FIG. 4 is a diagram showing the ability of the anti-LARC antibody to inhibit migration of peripheral blood mononuclear cells induced by the culture supernatant of the synovial cells derived from a rheumatoid arthritis patient.

FIG. 5 is a diagram showing the ability of the anti-CCR6 antibody to inhibit migration of peripheral blood mononuclear cells induced by the culture supernatant of the synovial cells derived from a rheumatoid arthritis-patient.

FIG. 6 shows the results of immunostaining to examine the expression of LARC protein in the synovial tissue of a rheumatoid arthritis patient and in the synovial tissue of an osteoarthritis patient.

FIG. 7 shows the results of in situ hybridization performed to study the expression of LARC mRNA in the synovial tissue of a rheumatoid arthritis patient.

FIG. 8 shows the results of immunostaining to examine infiltration of CCR6-positive cells into the synovial tissue of a rheumatoid arthritis patient.

FIG. 9 shows the results of in situ hybridization performed to study the expression of CCR6 mRNA in the synovial tissue of a rheumatoid arthritis patient.

FIG. 10 is a graph showing the results of real-time PCR to examine the time-dependency of the expression of LARC mRNA in the joints of mice with type II collagen-induced arthritis.

FIG. 11 is a graph showing the results of real-time PCR to examine the time-dependency of the expression of CCR6 mRNA in the joints of mice with type II collagen-induced arthritis.

FIG. 12 shows the results of immunostaining to examine infiltration of dendritic cells into synovial tissue of mice with type II collagen-induced arthritis.

FIG. 13 shows the results of RT-PCR to examine the time-dependency of the expression of LARC mRNA in the joints of rats with type II collagen-induced arthritis.

FIG. 14 shows the results of RT-PCR to examine the time-dependency of the expression of mRNA that is presumed to be a transcript for CCR6 in the joints of rats with type II collagen-induced arthritis.

FIG. 15 shows the results of immunostaining to examine the expression of LARC protein in the synovial tissue of rats with type II collagen-induced arthritis.

### BEST MODE FOR CARRYING OUT THE INVENTION

As used herein, the term therapeutic or prophylactic agent for rheumatoid arthritis refers to any pharmacological agent that can alleviate, treat, or prevent clinical symptoms of the disease when administered to patients suffering from rheumatoid arthritis.

The term LARC refers to a type of CC chemokine that has recently been discovered. LARC is mainly expressed in liver, lung, mesenteric tissues, lymphnodes, tonsil, and skin tissues, and has an ability to induce migration of immature dendritic cells, precursor cells of dendritic cells, B-lymphocytes, and memory T-lymphocytes. LARC is expressed in two different ways: constitutive expression observed in secondary lymphatic tissues except for spleen, and inductive expression in response to inflammatory stimuli. Thus, LARC is thought to act both to maintain homeostasis and to promote migration of cells to inflamed tissues (Hieshima et al., J. Biol. Chem., 1997, 272, 5846; Rossi et al., J. Immunol., 1997, 158, 1033; Hromas et al., Blood, 1997, 89, 3315; Power et al., J. Exp. Med., 1997, 186, 825; Cook et al., Immunity, 2000, 12, 495).

LARC-induced cell migration is mediated by the activation of LARC receptors, such as CCR6 chemokine receptor, which are expressed in immature dendritic cells, precursor cells of dendritic cells, B-lymphocytes, and memory T-lymphocytes (Baba et al., J. Biol. Chem., 1997, 272, 14893; Greaves et al., J. Exp. Med., 1997, 186, 837; Dieu et al., J. Exp. Med., 1998, 188, 373; Liao et al., J. Immunol., 1999, 162, 186; Bowman et al., J. Exp. Med., 2000, 191, 1303).

In general, one particular type of chemokine is known to bind to several types of chemokine receptors, rather than only one. Conversely, one particular type of chemokine receptor can bind to more than one type of chemokine. This suggests the presence of receptors other than CCR6 that can bind to LARC (Murdoch et al., Blood, 2000, 95, 3032).

It is known that immature dendritic cells first infiltrate into the synovium of rheumatoid arthritis. The infiltrated immature dendritic cells differentiate into mature dendritic cells in the synovium and present autoantigen to the etiologic T-lymphocytes to activate them. These etiologic T-lymphocytes subsequently serve to activate macrophages and B-lymphocytes. On the other hand, unlike rheumatoid arthritis because only a small number of dendritic cells are found in the synovial tissue of healthy people and osteoarthritis patients, T-lymphocytes also are not activated in the synovium. (Sato Kazuto, Igaku-no-ayumi, 1997, 182, 534).

Since dendritic cells play a key role in activating T-lymphocytes by presenting antigens, preventing infiltration of dendritic cells into the synovium can suppress activation of T-lymphocytes and thus activation of macrophages and B-lymphocytes (Thomas et al., Immunol. Today, 1996, 17, 559; Kamisaka et al., Mebio, 1995, November issue, 44; Thomas et al., Arthritis. Rheum., 1992, 35, 1455).

Thus, having an activity to prevent infiltration, differentiation, and activation of dendritic cells in rheumatoid joints, the therapeutic agent for rheumatoid arthritis according to the present invention can be used to treat or prevent rheumatoid arthritis at an early stage of the disease.

At a relatively late stage of the disease, B-lymphocytes form follicles in the synovium of rheumatoid arthritis patients. The B-lymphocytes are activated in these follicles and produce rheumatoid factors, which function as autoantibodies (Reparon Schuijt et al., Arthritis. Rheum., 1998, 41, 2211; Kim et al., J. Immunol., 1999, 162, 3053; Williams et al., Immunology, 1999, 98, 123). Rheumatoid factors have recently been suggested to be involved in the processes through which the disease progresses to its chronic stage. Thus, the therapeutic agent for rheumatoid arthritis in accordance with the present invention, which has an activity to prevent infiltration, differentiation, and activation of B-lymphocytes in rheumatoid joints, can be used to treat, and prevent progress of, rheumatoidarthritis (Kamisaka et al., Mebio, 1995, November issue, 44).

As used herein, the term “LARC inhibitor” refers to any substance that can inhibit biological activities of LARC. Examples include substances that can inhibit migration, differentiation, or activation of cells induced by LARC, and substances that can suppress expression of LARC RNA or LARC protein. Of these, the substances that can inhibit migration, differentiation, or activation of cells induced by LARC are preferred. More preferred LARC inhibitors are those obtained by evaluating candidate substances for the ability to inhibit LARC-induced cell migration.

Such “substances obtained through evaluation of candidate substances for the ability to inhibit LARC-induced cell migration” can be obtained by evaluating candidate substances for the ability to inhibit cell migration induced by a commercially available LARC protein or a LARC protein that is expressed in bacteria, yeast, mammalian cells, or insect cells. The “LARC protein” may be any LARC protein that exhibits biological activities of LARC and can be of any type and origin. For example, LARC proteins isolated from mammals, and LARC proteins obtained from cultured mammalian cells or supernatants of cultured mammalian cells can be used. Of these, LARC proteins present in the supernatant of cultured synovial cells derived from rheumatoid arthritis patients are preferred, while other proteins found in supernatants of cultured synovial cells derived from rheumatoid arthritis patients may also serve as a protein of interest.

Preferably, the “substances obtained through evaluation of candidate substances for the ability to inhibit LARC-induced cell migration” are obtained by comparing the ability of candidate substances to inhibit LARC-induced cell migration with that of a known anti-LARC antibody. Specifically, as will be described in Examples 3 and 4, it is desired that evaluation of candidate substances be done by using an anti-LARC antibody to assess the ability of the candidate substances to inhibit cell migration, although the substance may be of any type and origin, as long as it has been obtained by evaluating candidate substances for the ability to inhibit any biological activity of LARC.

For instance, such a substance may be obtained by evaluating candidate substances for the ability to inhibit a transient increase of intracellular calcium level caused by LARC, or for the ability to inhibit cell adhesion caused by LARC.

As used herein, the term “LARC inhibitor” includes LARC antagonists (competitive inhibitors) that can interrupt signal transduction mediated by LARC and thus can suppress biological activities of LARC.

The “LARC inhibitor” may be a substance of any type and origin as long as it can inhibit biological activities of LARC. For example, the LARC inhibitors may be chemicals, neutralizing antibodies against LARC, antisense DNA for a part of a LARC gene, or the like. Preferably, the LARC inhibitors are chemicals or neutralizing antibodies. Anti-LARC antibodies are particularly preferred.

As will be described in Examples 3 and 4, the anti-LARC antibodies are preferably antibodies capable of inhibiting LARC-induced cell migration, although antibodies of any type (e.g., monoclonal or polyclonal) and origin may be used, provided that they can inhibit any biological activity of LARC. While monoclonal antibodies derived from mammals are preferred, monoclonal antibodies for use in the present invention are not limited to those produced by hybridomas but may be those artificially modified, for example, to reduce antigenicity against humans.

The anti-LARC antibodies may be obtained in the following manner: Synthetic peptides synthesized on a common peptide synthesizer based on a part of the estimated amino acid sequence of LARC, or LARC proteins produced by bacteria, yeast, mammalian cells, or insect cells that are transformed using LARC-expressing vectors are purified using ordinary protein chemistry techniques. The obtained LARC peptides or LARC proteins are used as an immunogen to immunize animals such as mice, rats, hamsters, and rabbits. Antibodies (polyclonal antibodies) are then obtained from the serum of these animals.

Alternatively, anti-LARC antibodies may be obtained in the following manner: Lymphocytes collected from spleens or lymph nodes of immunized mice or rats are fused with myeloma cells to make hybridomas according to the method by Kohler and Milstein (Kohler et al., Nature, 1975, 256, 495) or the improved method of Kohler and Milstein method devised by Ueda et al., (Ueda et al., Proc. Natl. Acad. Sci. USA, 1982, 79, 4386). These hybridomas are used to produce monoclonal-antibodies.

Specifically, the anti-LARC monoclonal antibodies can be obtained through the following steps:

(A) immunization of mice with LARC protein;

(B) extirpation of spleens from the immunized mice, followed by separation of spleen cells;

(C) fusion of the separated spleen cells with mouse myeloma cells in the presence of a fusion-promoting agent (e.g., polyethylene glycol) according to the method described by Kohler et al.;

(D) culturing of hybridoma cells selected in a selective medium that does not permit the growth of unfused myeloma cells;

(E) selection of hybridoma cells that produce a desired antibody using techniques such as enzyme-linked immunosorbent assay (ELISA) and western blotting, followed by cloning of the cells using techniques such as limiting dilution-culture method; and

(F) culturing of the hybridoma cells that produce anti-LARC monoclonal antibody to harvest the monoclonal antibody.

Also, the antibody for use in the present invention may be any commercially available antibody that has the ability to neutralize the biological activities of human LARC. A preferred example is rabbit IgG anti-human LARC polyclonal antibody (available from Pepro Tech).

As used herein, the term “inhibitor of LARC receptors” refers to any substance that can inhibit biological activities of LARC receptors. Examples of the inhibitor include substances that can inhibit migration, differentiation, or activation of cells mediated by the activation of LARC receptors, or substances that can suppress expression of RNA or proteins of LARC receptors. Of these, the substances that can inhibit migration, differentiation, and activation of cells mediated by the activation of LARC receptors are preferred. The inhibitors found through evaluation of the ability of candidate substances to inhibit the LARC receptor-mediated cell migration are more preferred. While CCR6, a chemokine receptor, is preferred to serve as the LARC receptor of the present invention, the LARC receptor may be of any type and origin as long as it can bind LARC and this binding mediates migration, differentiation, and activation of the cells.

The “substance obtained through comparison of the ability of candidate substances to inhibit LARC receptors and that of anti-LARC receptor antibody” for use in the present invention is preferably obtained by comparing the ability of candidate substances to inhibit cell migration with that of a known antibody that can inhibit LARC receptors, as will be described in Example 5. However, such a substance may be of any type and origin, provided that it has been obtained through evaluation of candidate substances for the ability to inhibit biological activity of LARC receptors. For example, such substances may be obtained by evaluating candidate substances for the ability to inhibit the transient increase of intracellular calcium ion level mediated by the activation of LARC receptors and for the ability to inhibit cell adhesion mediated by the activation of LARC receptors.

Although anti-CCR6 antibody is preferred as the anti-LARC receptor antibody of the present invention, the anti-LARC receptor antibody may be of any type and origin, provided that it can serve as an antibody against any receptor that can bind LARC and can thus mediate migration, differentiation, and activation of cells.

As used herein, the term “inhibitor of LARC receptors” is meant to encompass “antagonists (competitive inhibitors) of LARC receptors” that can block signal transduction mediated by the activation of LARC receptors and can thus suppress biological activity of LARC receptors.

Such an inhibitor of LARC receptors may be of any type and origin as long as it can inhibit biological activities of LARC receptors. For example, the LARC receptor inhibitors may be chemicals, neutralizing antibodies against LARC receptors, antisense DNA for a part of LARC receptor genes, or the like. Preferably, the LARC receptor inhibitors are chemicals or neutralizing antibodies. Anti-LARC receptor antibodies are particularly preferred.

The anti-LARC receptor antibodies are preferably antibodies that can inhibit cell migration mediated by the activation of LARC receptors, although antibodies of any type (e.g., monoclonal or polyclonal) and origin may be used, provided that they can inhibit biological activities of LARC receptors. While monoclonal antibodies derived from mammals are preferred, monoclonal antibodies for use in the present invention are not limited to those produced by hybridomas but may be those artificially modified, for example, to reduce antigenicity against humans.

The anti-LARC receptor antibodies can be obtained in the following manner: Synthetic peptides synthesized on a common peptide synthesizer based on a part of the estimated amino acid sequence of LARC receptor, or LARC receptor proteins produced by bacteria, yeast, mammalian cells, or insect cells that are transformed using LARC receptor-expressing vectors are purified using ordinary protein chemistry processes. The obtained LARC receptor peptides or LARC receptor proteins are used as an immunogen to immunize animals such as mice, rats, hamsters, or rabbits. Antibodies (polyclonal antibodies) are obtained from the serum of these animals.

Alternatively, the anti-LARC receptor antibodies may be obtained in the following manner: Lymphocytes collected from spleens or lymph nodes of immunized mice or rats are fused with myeloma cells to make hybridomas according to the method by Kohler and Milstein (Kohler et al., Nature, 1975, 256, 495) or the improved method of the Kohler and Milstein method devised by Ueda et al., (Ueda et al., Proc. Natl. Acad. Sci. USA, 1982, 79, 4386). These hybridomas are used to produce monoclonal antibodies.

Specifically, anti-LARC receptor antibodies can be obtained through the following steps:

(A) immunization of mice with LARC receptor proteins;

(B) extirpation of spleens from the immunized mice, followed by separation of spleen cells;

(C) fusion of the separated spleen cells with mouse myeloma cells in the presence of a fusion-promoting agent (e.g., polyethylene glycol) according to the methods described by Kohler et al.;

(D) culturing of hybridoma cells selected in a selective medium that does not permit the growth of unfused myeloma cells;

(E) selection of hybridoma cells that produce a desired antibody using techniques such as enzyme-linked immunosorbent assay (ELISA) and western blotting, followed by cloning of the cells using techniques such as limiting dilution-culture method; and

(F) culturing of hybridoma cells that produce anti-LARC receptor monoclonal antibody and harvest the monoclonal antibody.

The antibody for use in the present invention may be any commercially available antibody that has an ability to neutralize the biological activities of human LARC receptors. A preferred example is mouse IgG2b anti-human CCR6 monoclonal antibody (available from R&D Systems).

Furthermore, the therapeutic or prophylactic agent for rheumatoid arthritis can be screened for by evaluating candidate substances for the ability to inhibit cell migration induced by a commercially available LARC protein or by a LARC protein that is forcibly expressed in bacteria, yeast, mammalian cells, or insect cells. The LARC protein to be used for that purpose may be any LARC protein that exhibits biological activities of LARC and maybe of any type and origin. For example, LARC proteins isolated from tissues of mammals, and LARC proteins obtained from cultured mammalian cells or supernatants of cultured mammalian cells can be used. Of these, LARC proteins present in supernatants of cultured synovial cells derived from rheumatoid arthritis patients are preferred, while other proteins may also serve as a protein of interest. More preferably, the therapeutic or prophylactic agent for rheumatoid arthritis is screened for by comparing the ability of candidate substances to inhibit LARC with that of a known anti-LARC antibody or by comparing the ability of candidate substances to inhibit a LARC receptor with that of a known anti-LARC receptor antibody. For example, the therapeutic or prophylactic agent may be obtained by evaluating candidate substances for the ability to inhibit the transient increase of intracellular calcium ion level or for the ability to inhibit cell adhesion. Preferably, this screening method is used to screen chemicals and antibodies, although it may be used to screen other substances. The cells used in the screening process may be cells of any type and origin that are expressing,LARC receptor at any expression level. Preferred examples of the cells include cells genetically engineered to express LARC receptors such as CCR6, B-lymphocytes, memory T-lymphocytes, immature dendritic cells, and precursor cells of dendritic cells.

As used herein, the term “substance obtained by partially altering LARC polypeptides” is meant to encompass any substance that can bind to LARC receptors but lacks the biological activities inherent to LARC proteins. Preferred examples of such substances include those obtained by modifying, deleting, or fragmenting LARC proteins either physically, chemically or pharmacologically, although substances of any type and origin maybe used. Preferred examples of the substances include those obtained by modifying or deleting an adjacent region of the N-terminal of LARC, the region considered as a key element for signal transduction mediated by LARC. More preferred are the substances obtained by modifying or deleting the region extending from the N-terminal of LARC to the two- cysteine residues conserved among CC chemokines.

As used herein, the term “substance obtained by genetically altering LARC gene” is meant to encompass any variant of LARC protein obtained by genetically modifying, deleting, or altering LARC gene and synthesizing from the altered gene. Preferably, the variants of LARC proteins synthesized from the altered gene are capable of binding to LARC receptors but lack the biological activities inherent to LARC proteins. Preferred examples of the substance include those obtained by modifying or deleting an adjacent region of the N-terminal of LARC proteins, the region considered as a key element for signal transduction by LARC. More preferred are those obtained by modifying or deleting the region extending from the N-terminal to the two-cysteine residues conserved among CC chemokines. More specifically, the regions of LARC conserved among different biological species are genetically altered, although the substance may be of any type and origin.

Typically, model animals of rheumatoid arthritis play an important role as means for screening for therapeutic or prophylactic agents for rheumatoid arthritis. While preferred “models of rheumatoid arthritis” for use in the present invention are type II collagen-induced arthritis (CIA mice) in mice or type II collagen-induced arthritis in rats (CIA rats), model animals of any type and origin may be used, provided that they can be used to establish a disease model. Examples of the disease model established in animals include rat-adjuvant induced model, arthritis induced in rabbits, MRL mice, arthritis induced with anti-type II collagen antibody, TNF-a transgenic animals, mice with severe combined immunodeficiency (SCID), and arthritis induced in monkeys. In the present invention, as will be described in Examples 10 through 15, the association of LARC, CCR6 and related cells with the disease state has been clearly shown in typical model animals of rheumatoid arthritis as in the synovium of rheumatoid arthritis. Accordingly, the model animals of rheumatoid arthritis can be used in screening LARC inhibitors, LARC receptor inhibitors, antagonists of LARC, and antagonists of LARC receptors, in order to obtain the therapeutic or prophylactic agent for rheumatoid arthritis.

As used herein, the term “LARC inhibitors obtained through evaluation using model animals of rheumatoid arthritis” refers to any inhibitor that can inhibit biological activities of LARC in mammals. Examples of such inhibitors include substances that can inhibit LARC-induced migration, differentiation, and activation of cells, and substances that can suppress expression of LARC RNA or expression of LARC proteins. Among these substances, those that can suppress infiltration of cells into the synovium in model animals of rheumatoid arthritis are preferred, although the inhibitor may be of any type and origin, provided that it can alleviate any of the following symptomatic properties:

(1) the level of antibodies against type II collagen in blood;

(2) edema, or paw volume;

(3) destruction of bone or cartilage;

(4) inflammation in legs, tails, or nostrils;

(5) erythrocyte sedimentation rate;

(6) increase in the leucocyte count in peripheral blood;

(7) variation in weight of organs;

(8) acute phase reactive proteins such as fibrinogen and C-reactive proteins (CRP);

(9) autoantibody titer of antibodies against DNA or the like;

(10) survival rate; and

(11) hyperplasia of synovium.

More preferably, anti-LARC antibodies are used.

The term “LARC inhibitors obtained through evaluation using model animals of rheumatoid arthritis” as used herein further encompasses “LARC antagonists (competitive inhibitors)” that can block signal transduction mediated by LARC and thus-can suppress biological activities of LARC.

As used herein, the term “LARC receptor inhibitors obtained through evaluation using model animals of rheumatoid arthritis” refers to any inhibitor that can inhibit biological activities of LARC receptors in mammals. Examples of such inhibitors include substances that can inhibit migration, differentiation, and activation of cells mediated by LARC receptors, and substances that can suppress expression of LARC receptor RNA or LARC receptor proteins. While CCR6 chemokine receptor is preferred as the LARC receptor, the LARC receptor may be of any type and origin as long as it can bind LARC and this binding mediates about migration, differentiation, and activation of cells.

Among various “LARC receptor inhibitors that are obtained through evaluation using model animals of rheumatoid arthritis”, those that can suppress infiltration of cells into the synovium of model animals of rheumatoid arthritis are preferred, although the inhibitor may be of any type and origin, provided that it can alleviate any of the following symptomatic properties:

(1) the level of antibodies against type II collagen in blood;

(2) edema, or paw volume;

(3) destruction of bone or cartilage;

(4) inflammation in legs, tails, or nostrils;

(5) erythrocyte sedimentation rate;

(6) increase in the leucocyte count in peripheral blood;

(7) variation in weight of organs;

(8) acute phase reactive proteins such as fibrinogen and C-reactive proteins (CRP);

(9) autoantibody titer of antibodies against DNA or the like;

(10) survival rate; and

(11) hyperplasia of synovium.

More preferred are anti-LARC receptor neutralizing antibodies and anti-CCR6 antibodies.

The term “LARC receptor inhibitors obtained through evaluation using model animals of rheumatoid arthritis” as used herein further encompasses “LARC receptor antagonists (competitive inhibitors)” that can block signal transduction mediated by the activation of LARC receptors and thus can suppress biological activities of LARC receptors.

In addition, the above-described model animals of rheumatoid arthritis may be used to screen for the therapeutic or prophylactic agent for rheumatoid arthritis. In a preferred method, suppression of the cell infiltration into the synovium of the model animals of rheumatoid arthritis is evaluated. This is preferably done by evaluating by how much the following symptomatic properties are alleviated:

(1) the level of antibodies against type II collagen in blood;

(2) edema, or paw volume;

(3) destruction of bone or cartilage;

(4) inflammation in legs, tails, or nostrils;

(5) erythrocyte sedimentation rate;

(6) increase in the leucocyte count in peripheral blood;

(7) variation in weight of organs;

(8) acute phase reactive proteins such as fibrinogen and C-reactive proteins (CRP);

(9) autoantibody titer of antibodies against DNA or the like;

(10) survival rate; and

(11) hyperplasia of synovium.

In a more preferred method, the therapeutic or prophylactic agent for rheumatoid arthritis is screened for by comparing the ability of candidate substances to inhibit LARC with that of a known anti-LARC antibody, or by comparing the ability of candidate substances to inhibit LARC receptor with that of a known anti-LARC receptor antibody. Preferably, this screening method is used to screen chemicals and neutralizing antibodies, although it may be used to screen other substances.

As will be described later, Examples 1 and 2 indicate that the synovial cells derived from rheumatoid arthritis patients are capable of expressing LARC in vitro.

The data obtained in Example 3 indicate that LARC produced by the synovial cells derived from rheumatoid arthritis patients are capable of inducing migration of lymphocytes and this cell migration is inhibited by anti-LARC antibody, which serves as a LARC inhibitor.

Examples 4 and 5 indicate that LARC produced by the synovial cells derived from rheumatoid arthritis patients are capable of inducing migration of peripheral blood mononuclear cells and this cell migration is inhibited by anti-LARC antibody, which serves as a LARC inhibitor, and anti-CCR6 antibody, which serves as a CCR6 inhibitor. Since cell infiltration into the synovial tissue of rheumatoid joints takes place as a result of chemokine-induced cell migration, it is expected that the “LARC inhibitors” and the “LARC receptor inhibitors” can act to suppress infiltration of B-lymphocytes, dendritic cells, and memory T-lymphocytes into the synovium.

Examples 6 through 9 are analyses of:

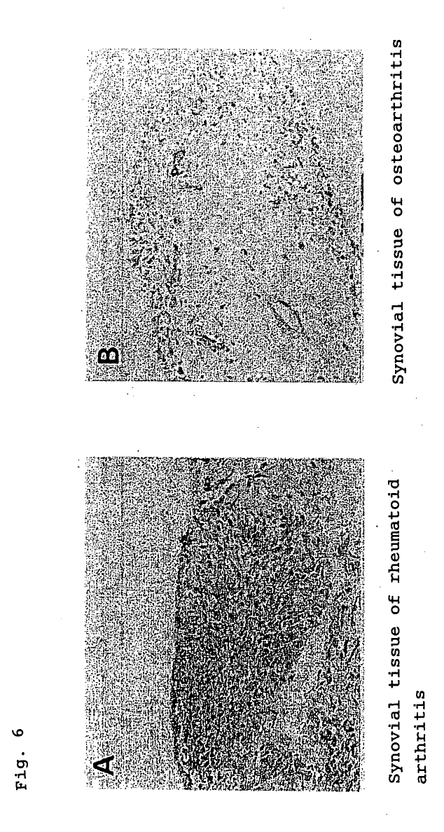

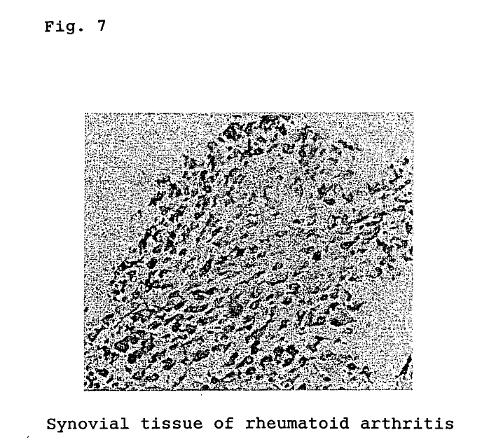

(1) expression of LARC (FIGS. 6 and 7), and

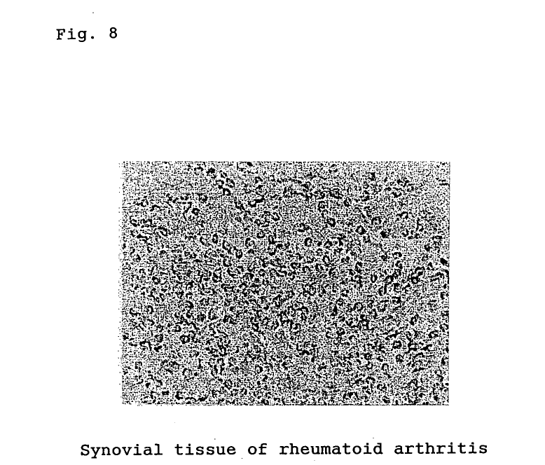

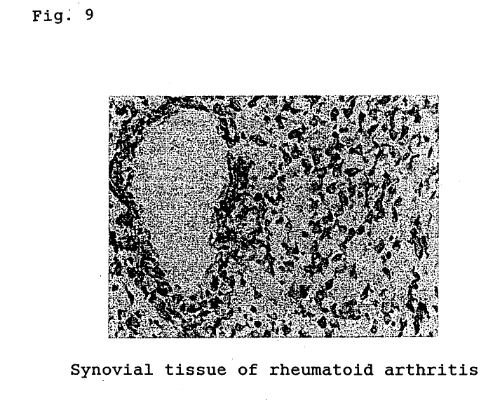

(2) localization of CCR6-positive cells (FIGS. 8 and 9) in the synovial tissue of rheumatoid arthritis.

Regarding (1) above, the expression of LARC was found in the synovial tissue of rheumatoid arthritis, whereas no LARC was detected in the synovial tissue of osteoarthritis. Regarding (2) above, CCR6-positive cells were localized in the synovial tissue of rheumatoid arthritis. The results of (1) and (2) together indicate that LARC is expressed only in the synovium of rheumatoid arthritis that have been infiltrated with B-lymphocytes, dendritic cells, and memory T-lymphocytes, whereas LARC is not expressed in the synovium of osteoarthritis that have been little infiltrated with B-lymphocytes, dendritic cells and memory T-lymphocytes.

In Examples 10 and 11, the time-dependency of expression of mRNA for LARC (Example 10) and CCR6 (Example 11) were analyzed in the affected joints in mice with type II collagen-induced arthritis, which served as a model animal of rheumatoid arthritis. The results show that the expression levels of LARC and CCR6 showed an increase in the arthritic mice and the peak of expression coincided with the time at which cell infiltration into the synovium was most significant. Since infiltration of dendritic cells was observed in the synovium of the arthritic joints (Example 12), these results indicate that LARC- or LARC receptor-induced cell infiltration into the synovium cause the disease of not only animal models but also rheumatoid arthritis.

In Examples 13 and 14, the time-dependency of expression of mRNA for LARC (Example 13) and the time-dependency of expression of mRNA that is presumed to be a transcript for CCR6 (Example 14) were analyzed in the affected joints of rats with type II collagen-induced arthritis, which served as a model animal of rheumatoid arthritis. The results indicate that the expression levels of LARC and CCR6 each showed an increase in the arthritic rats. While rat CCR6 has not been identified yet, many tests indicate that mRNA analyzed in Example 14 is the mRNA for the rat CCR6.

The rat CCR6 can be obtained in the following manner: An expressed sequence tag (EST) of rat CCR6 is obtained from the EST library at the National Center for Biotechnology Information, USA (NCBI) database. This sequence is used to perform Rapid Amplification of cDNA Ends (RACE method). One example of the EST for rat CCR6 is NCBI database accession No. AI045155.

In Example 15, the expression of LARC protein in the synovium of knee joints of rats with type II collagen-induced arthritis was examined. The presence of LARC protein was observed in endothelial cells of a blood vessel newly formed in the synovial tissue.

Accordingly, the results of Examples 10 through 15 indicate that LARC and LARC receptors are also involved in the onset of the disease in the model animals of rheumatoid arthritis. This implies the possibility that administration of a “LARC inhibitor” or a “LARC receptor inhibitor” to model animals of rheumatoid arthritis can treat the disease. Preferably, the “LARC inhibitors” are anti-LARC antibodies and the “LARC receptor inhibitors” are anti-CCR6 antibodies although use of other substances may also be contemplated.

The “LARC inhibitors” or the “LARC receptor inhibitors” may be administered to model animals of rheumatoid arthritis by following procedures used in successful known experiments in which antagonist of MCP-1, anti-RANTES antibody, or anti-IL-1 antibody was administered to model animals of rheumatoid arthritis (Barnes et al. , J. Clin . Invest., 1998, 101, 2910; van de Lo et al., Arthritis. Rheum., 1995, 38, 164; Gong et al., J. Exp. Med., 1997, 186, 131). Preferred routes of administration include intraperitoneal, intravenous, and intraarticular, although administration through other routes may also be contemplated. Although the preferred dosage may vary depending on properties and neutralizing ability of a particular “LARC inhibitor” or a particular “LARC receptor inhibitor”, a dosage of 0.5 mg/animal to5 mg/animal is preferred.

The results of Examples suggest that LARC or LARC receptors are responsible for inducing infiltration of B-lymphocytes, dendritic cells, and memory T-lymphocytes, which is believed to be the cause of rheumatoid arthritis, and that inhibition of the reactions mediated by LARC or by the activation of LARC receptors can provide a novel treatment for the disease.

Preferably, the LARC inhibitors, the LARC receptor inhibitors, the LARC antagonists, or the LARC receptor antagonists for use in the present invention may be mixed with a pharmacologically acceptable carrier to be used as the therapeutic or prophylactic agent of the present invention.

The pharmacologically acceptable carrier may be those used as a vehicle, which will be described later. While the mixing ratio of LARC inhibitor, LARC receptor inhibitor, antagonist of LARC, or antagonist of LARC receptor to the carrier is determined based on a desired dosage of the active ingredient, it can vary considerably and thus is not limited to a particular range. Typically, an inhibitor of MCP-1, which belongs to the same chemokine sub-group as LARC, i.e., CC chemokines, is mixed in a composition in an amount of 1 to 70 wt %, preferably 5 to 50 wt %. Using a proper vehicle in a known manner, the obtained composition is further prepared into the forms of soft capsule, hard capsule, tablet, pellet, powder, suspension, solution, syrup, or the like for use as an ingestion, an injection, a suppository, or a topical preparation.

Examples of such vehicles include vegetable oils (e.g., corn oil, cottonseed oil, coconut oil, almond oil, peanut oil, olive oil, and the like), oily esters such as middle-chained fatty acid glycerides, mineral oils, glycerol esters such as tricaprilin and triacetin, alcohols such as ethanol, saline, propylene glycol, polyethylene glycol, Vaseline, animal fats and oils, cellulose derivatives (e.g., crystalline cellulose, hydroxypropylcellulose, hydroxypropylmethylcellulose, methylcellulose), poly(vinylpyrrolidone), cyclodextrin, dextrin, lactose, mannitol, sorbitol, and starch.

The dosage of the agent, though it may vary depending on the severity of the disease and age of patients, is about 0.01 mg/day/patient to about 1000 mg/day/patient, preferably 1 mg/day/patient to 200 mg/day/patient. Preferably, the preparation is formulated to satisfy these conditions.

### EXAMPLES

While the present invention will now be specifically described with reference to Examples, they are intended to be only illustrative rather than restrictive and thus do not limit the scope of the invention in any way.

### Example 1

### Establishing Synovial Cells Derived from a Rheumatoid Arthritis Patient and Expression of LARC mRNA in the Synovial Cells.

(1) Preparation of Synovial Cell

Synovial tissue was obtained during surgical procedures of joint replacement of a rheumatoid arthritis patient. The synovial tissue was cut into strips with scissors and then enzymatically digested by treating with a 0.2 mg/mL collagenase solution (available from Worthington Biochemical Corp.,) at 37° C. for 1 hour. The digests were passed through a mesh to obtain single cells. The isolated cells were collected in a test tube and were washed with Dulbecco's modified Eagle's medium (DMEM) (available from GIBCO BRL) containing 10% fetal bovine serum (available from JRH Bioscience), 10 mM HEPES, 100 units/mL penicillin, and 100 μg/mL streptomycin. The cells were then suspended in the above-prepared medium and cultured overnight at 37° C. under 5% CO₂. After culturing, unbound cells were removed to obtain synovial cells for use in the present invention. The synovial cells were cultured for 5 to 8 passages before used in the following experiments.

(2) Expression of LARC mRNA in Synovial Cells

The synovial cells obtained above were cultured in the above-described medium for several days. Once the cells reached subconfluence, 1 ng/mL recombinant human interleukin-1β (IL-1β) (available from Cambridge) or 1 ng/mL recombinant human tumor necrosis factor-α (TNF-a) (available from Cambridge) was added to the culture and the cells were stimulated for 1 hour.

RT-PCR and southern hybridization were performed to examine in the expression level of LARC in the synovial cells. Total RNA was extracted from the synovial cells with ISOGEN (available from Nippon Gene) and was reverse-transcribed using cDNA synthesis kit (available from Takara) to synthesize cDNA. PCR was performed in a total volume of 25 μL containing 0.5 μM of each of sense primer and antisense primer for each target gene, 2.5U Taq DNA polymerase (available from Takara), 5 μL cDNA, 0.2 mM dNTP mixture, and 1.5 mM MgCl₂.

The primers used were: for LARC:

5′-TTGGATCCTGCTGCTACTCCACCTCTG-3′ and

5′-TTCTCGAGTATATTTCACCCAAGTCTGTTTT-3′; for beta 2-microglobulin(β2m): 5′-TTCTGGCCTGGAGGGCATCC-3′ and

5′-ATCTTCAAACCTCCATGATG-3′.

The reaction conditions were as follows: 30 cycles of 1) denaturation (1 min at 95° C.), 2) annealing (30 sec at 52° C.), and 3) extension (30 sec at 72° C.) for amplification of the LARC gene, and 25 cycles for the β2m gene. A portion of the PCR products was electrophoresed on 2% agarose gel (available from Nippon Gene) containing 0.5 μg/mL ethidium bromide (available from Sigma). Electrophoresis image analysis was performed by southern hybridization using DIG-High Prime DNA Labeling and Detection Kit (available from Roche Diagnostics GmbH).

The probes used were: for LARC:

5′-GATGTCACAGCCTTCATTGG-3′; for beta 2-microglobulin(β2m):

5′-ACACGGCAGGCATACTCATC-3′.

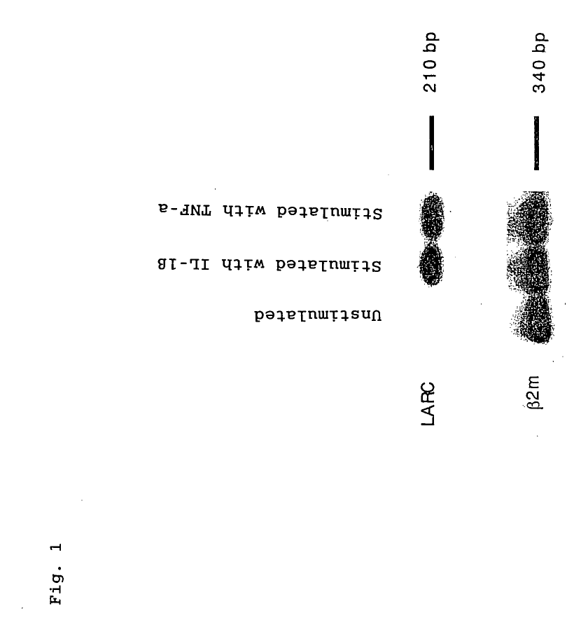

Shown in FIG. 1 are the results of RT-PCR and southern hybridization, indicating the expression levels of LARC mRNA in the synovial cells. As shown, LARC mRNA was expressed in synovial cells one-hour after the treatments with IL-1β or TNF-α, whereas unstimulated synovial cells did not express LARC mRNA. Since the expression levels of β2 m mRNA, serving as an internal standard, were the same for every sample, these results indicate that the synovial cells express LARC mRNA in response to the stimuli of IL-1β or TNF-a.

### Example 2

### Expression of LARC Protein in the Synovial Cells Derived from a Rheumatoid Arthritis Patient

Synovial cells derived from a rheumatoid arthritis patient (available from CELL APPLICATIONS) were cultured for 24 hours in a 4-well chamber slide (available from Iwaki Glass) in DMEM medium (available from GIBCO BRL) containing 10% fetal bovine serum (available from GIBCO BRL), 10 mM HEPES, 100 units/mL penicillin, and 100 μg/mL streptomycin. Subsequently, 10 ng/mL IL-1β (available from R&D Systems) and 10 ng/mL TNF-a (available from R&D Systems) were added to the culture, and the culture was stimulated for 24 hours.

After the culture, the culture medium was discarded and the chambers were removed. PBS (0.1M) was added to each well and the cells were gently washed. The cells were then fixed with PBS containing 4% paraformaldehyde at room temperature for 10 minutes. This was followed by washing three times with PBS at room temperature and then treatment for 5 minutes with PBS containing 0.2% Triton X-100 (available from Sigma). The slides were then washed five times with PBS, air-dried with a drier, and then completely dried in an incubator. The following procedures were performed at room temperature. After drying, endogenous peroxidase was removed with hydrogen peroxide, followed by washing twice with PBS. The cells were treated with PBS containing 1% non-fat dry milk (available from Wako Jun-yaku) overnight to block non-specific antibody binding. The slides were then washed twice with PBS and incubated with goat IgG anti-human LARC polyclonal antibody (2 μg/mL) (available from R&D Systems) for 1 hour. After washing three times with PBS, the cells were incubated with biotinylated rabbit anti-goat IgG polyclonal antibody (2 μg/mL)(available from DAKO) for 1 hour. The slides were then washed three times with PBS, incubated with peroxidase-streptavidin (available from DAKO) for 1 hour, and again washed three times with PBS. Finally, diaminobenzidine (available from DAKO) was applied to the slides, and the cells were counterstained with hematoxylin and observed under a microscope.

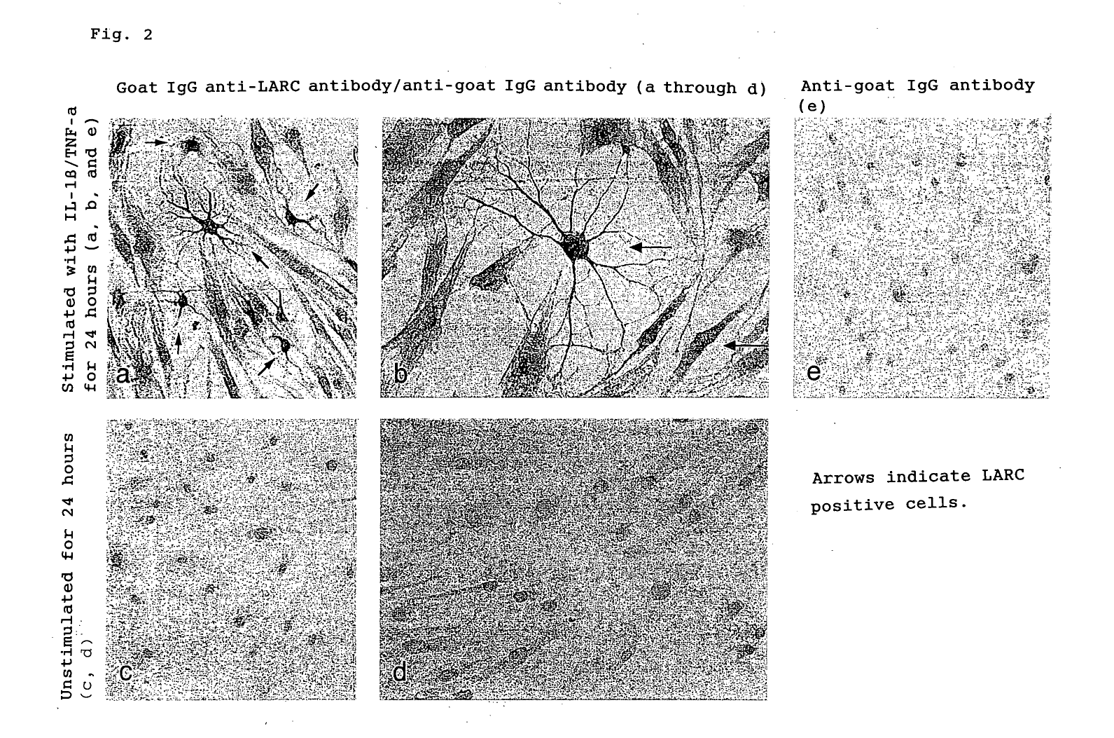

Shown in FIG. 2 are the results of the immunostaining to examine expression of LARC protein in the synovial cells. As shown, LARC protein was expressed in the synovial cells stimulated with IL-1β and TNF-a (FIGS. 2a and 2b). In comparison, as can be seen in FIG. 2e, no positive cell was detected when only biotinylated rabbit anti-goat IgG polyclonal antibody was applied. Also, LARC protein was not expressed in the unstimulated synovial cells (FIGS. 2c and 2d). Collectively, these observations suggest that when stimulated with IL-1β and TNF-a, the synovial cells express LARC-protein as well as LARC mRNA.

### Example 3

### Inhibition of Lymphocyte Migration by Anti-LARC Neutralizing Antibody (Measurement of the Ability of Lymphocytes to Migrate in Response to LARC Produced by the Synovial Cells Derived from a Rheumatoid Arthritis Patient)

The synovial cells used in Example 2 were incubated in DMEM medium (available from GIBCO BRL) containing 0.5% fetal bovine serum (available from GIBCO BRL) for 24 hours. Subsequently, the culture medium was replaced with serum-free DMEM. 10 ng/mL IL-1β (available from R&D Systems) and 10 ng/mL TNF-a (available from R&D Systems) were then added to the culture, and the culture was subsequently stimulated for 24 hours. After the culture, the culture supernatant was collected and stored at −80° C. until measurement of chemotactic response of the lymphocytes.

The lymphocytes used in the measurement of chemotactic response were prepared from 50 mL of peripheral blood collected from a healthy individual with a vacuum blood collection tube containing heparin. The assay was performed in the following manner. To the collected blood, an equal amount of PBS was added, and the mixture was carefully overlaid on top of Ficoll-Paque (available from Pharmacia) placed in a test tube. The test tube was centrifuged for 30 minutes at 1500 rpm at 20° C. to collect a layer of mononuclear cells. To remove CD14-positive monocytes from the obtained mononuclear cells, magnetic beads coated with anti-CD14 antibody (available from DYNAL) were added. After 1 hour incubation period with the magnetic beads at 4° C., a special magnet was used to attract the beads and unbound cells were collected to serve as the lymphocytes for the measurement. The lymphocytes were suspended in DMEM containing 0.1% fetal bovine serum albumin (BSA) at a concentration of 5×10⁶ cells/mL. To the suspension, one-thousandth as much of 10 mM 5 (and 6)-carboxyfluorescein diacetate succinimidyl ester (CFSE) (available from Wako Jun-yaku) was added as a fluorescent reagent, and the suspension was incubated for 20 minutes at room temperature.

The chemotactic response of the lymphocytes was measured on a 96-well microplate (available from Neuro Probe) equipped with a polyvinylpyrrolidone-free filter with the pore size of 5 μm. The suspension of CFSE-labeled lymphocytes with a cell concentration of 5×10⁶ cells/mL was added to the plate in an amount of 20 μL/well to serve as standards. The following test solutions were added to the wells on the plate in an amount of 30 μL/well: 1) the above-described supernatant of the cell culture; 2) the same supernatant incubated with rabbit IgG anti-human LARC polyclonal antibody (30 μg/mL) (available from Pepro Tech) for 30 minutes at room temperature; and 3) the same supernatant incubated with rabbit IgG control antibody (30 μg/mL) (available from DAKO) for 30 minutes at room temperature. The filter was then attached to the plate, and the plate was incubated in a 5% CO₂ incubator for 15 minutes. The suspension of CFSE-labeled lymphocytes with a cell concentration of 5×10⁶ cells/mL was added to the wells other than the standard wells on top of the filter. Subsequently, the plate was incubated in the 5% CO₂ incubator for another 3 hours. Following removal of the cells remaining on the filter, the filter was removed and the fluorescence intensity was measured for each well on Cytoflour 2000 (available from Millipore).

The chemotactic response of the lymphocytes was determined by the following equation: The chemotactic response (%)=(fluorescence intensity detected in the test well/fluorescence intensity detected in the standard well)×100

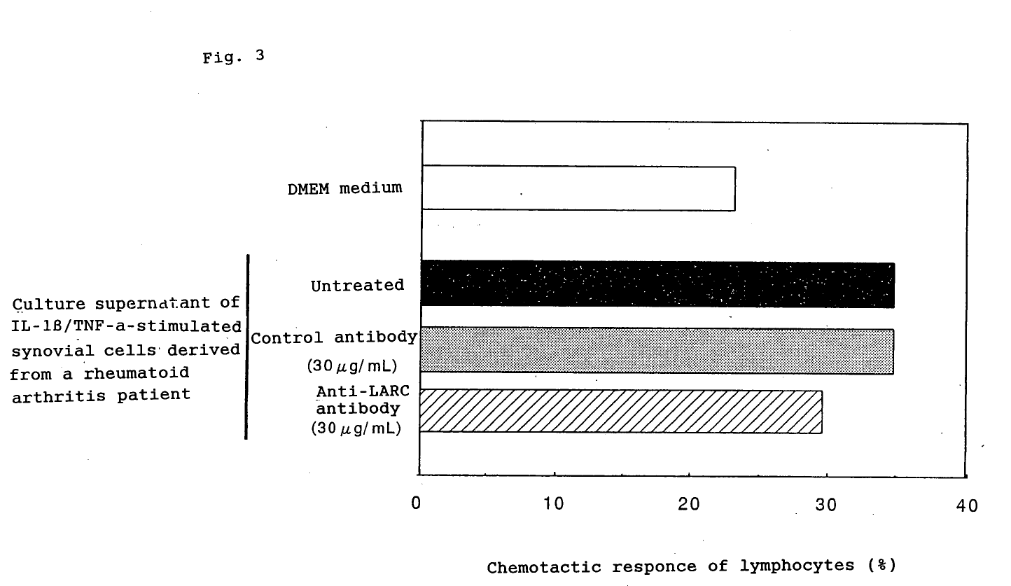

Shown in FIG. 3 are the results indicating the chemotactic response of the lymphocytes toward LARC produced by the synovial cells. As shown, 34.8% of the lymphocytes exhibited chemotaxis toward the culture supernatant of the synovial cells stimulated with IL-1β and TNF-a for 24 hours. In comparison, the same supernatant incubated with 30 μg/mL rabbit IgG anti-human LARC polyclonal antibody, a LARC inhibitor, elicited chemotaxis in 29.7% of the lymphocytes. This corresponds to a 5.1% decrease in the chemotactic response of the lymphocytes as compared to the untreated supernatant. Since the supernatant incubated with rabbit IgG control antibody elicited chemotaxis in 34.9% of the lymphocytes, which is comparable to the case of untreated supernatant, these results suggest that rabbit IgG anti-human LARC polyclonal antibody acts to neutralize LARC present in the culture supernatant of the synovial cells and thus to reduce the chemotactic response of lymphocytes. Accordingly, it has been shown that LARC produced by the synovial cells are capable of inducing migration of lymphocytes. Also, it has been shown that LARC inhibitors act to inhibit the cell migration toward LARC.

### Example 4

### Inhibition of Migration of Peripheral Blood Mononuclear Cells by Anti-LARC Neutralizing Antibody (Measurement of the Ability of Peripheral Blood Mononuclear Cells to Migrate in Response to LARC Produced by the Synovial Cells Derived from a Rheumatoid Arthritis Patient)

The synovial cells used in Example 2 were incubated in DMEM medium containing 0.5% fetal bovine serum for 24 hours. Subsequently, the culture medium was replaced with serum-free DMEM. 10 ng/mL IL-1β and 10 ng/mL TNF-a were then added to the culture, and the culture was subsequently stimulated for 24 hours. After the culture, the culture supernatant was collected and stored at −80° C. until measurement of the chemotactic response of the peripheral blood mononuclear cells.

The peripheral blood mononuclear cells used in the measurement of chemotactic response were prepared from 50 mL of peripheral blood collected from a healthy individual with a vacuum blood collection tube containing heparin. The assay was performed in the following manner. To the collected blood, an equal amount of PBS was added, and the mixture was carefully overlaid on top of Ficoll-Paque (available from Pharmacia) placed in a test tube. The test tube was centrifuged for 30 minutes at 1500 rpm at 20° C. to collect a layer of mononuclear cells. The mononuclear cells were suspended in DMEM containing 0.1% fetal bovine serum albumin (BSA) at a concentration of 5×10⁶ cells/mL. To the suspension, one-thousandth as much of 10 mM 5 (and 6)-carboxyfluorescein diacetate succinimidyl ester (CFSE) (available from Wako Jun-yaku) was added as a fluorescent reagent, and the suspension was incubated for 20 minutes at room temperature.

The chemotactic response of the mononuclear cells was measured on a 96-well microplate (available from Neuro Probe) equipped with a polyvinylpyrrolidone-free filter with the pore size of 5 μm. The suspension of CFSE-labeled peripheral blood mononuclear cells with a cell concentration of 5×10⁶ cells/mL was added to the plate in an amount of 20 μL/well to serve as standards. The following test solutions were added to the wells on the plate in an amount of 30 μL/well: 1) the above-described supernatant of the cell culture; 2) the same supernatant incubated with rabbit IgG anti-human LARC polyclonal antibody (30 μg/mL) for 30 minutes at room temperature; and 3) the same supernatant incubated with rabbit IgG control antibody (30 μg/mL) (available from DAKO) for 30 minutes at room temperature. The filter was then attached to the plate, and the plate was incubated in a 5% CO₂ incubator for 15 minutes. The suspension of CFSE-labeled peripheral blood mononuclear cells with a cell concentration of 5×10⁶ cells/mL was added to the wells other than the standard wells on top of the filter. Subsequently, the plate was incubated in the 5% CO₂ incubator for another 4 hours. Following removal of the cells remaining on the filter, the filter was removed and the fluorescence intensity was measured for each well on Cytoflour 2000 (available from Millipore).

The chemotactic response of the peripheral blood mononuclear cells was determined by the following equation: The chemotactic response (%)=(fluorescence intensity detected in the test well/fluorescence intensity detected in the standard well)×100

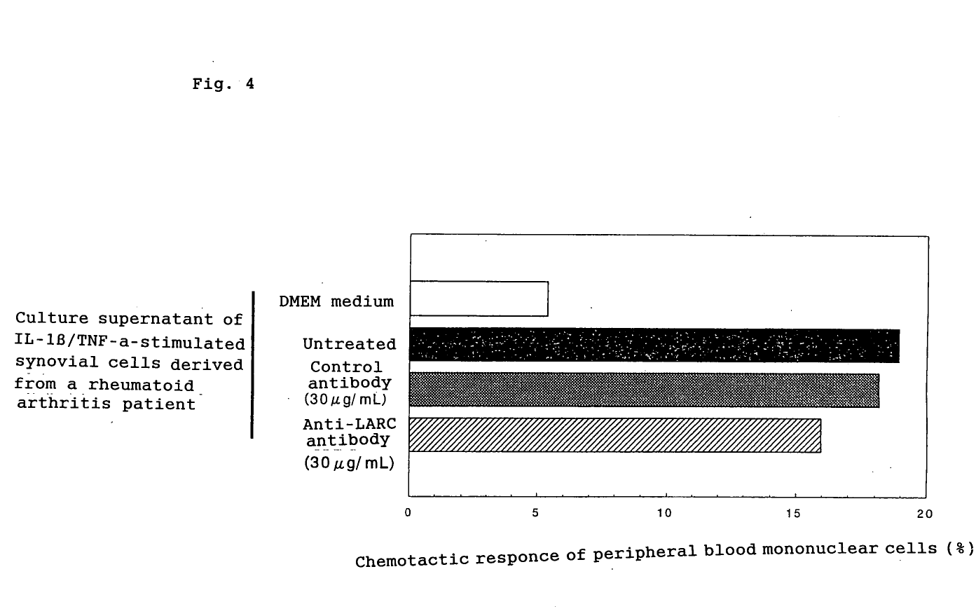

Shown in FIG. 4 are the results indicating the chemotactic response of the mononuclear cells toward LARC produced by the synovial cells. As shown, 18.9% of the mononuclear cells exhibited chemotaxis toward the culture supernatant of the synovial cells stimulated with IL-1β and TNF-a for 24 hours. In comparison, the same supernatant incubated with 30 μg/mL rabbit IgG anti-human LARC polyclonal antibody, a LARC inhibitor, elicited chemotaxis in 15.9% of the mononuclear cells. This corresponds to a 0.3.0% decrease in the chemotactic response of the mononuclear cells as compared to the untreated supernatant. Since the supernatant incubated with rabbit IgG control antibody elicited chemotaxis in 18.2% of the mononuclear cells, which is comparable to the case of untreated supernatant, these results suggest that rabbit IgG anti-human LARC polyclonal antibody acts to neutralize LARC present in the culture supernatant of the synovial cells and thus reduce the chemotactic response of mononuclear cells. Accordingly, it has been shown that LARC produced by synovial cells are capable of inducing migration of mononuclear cells. Also, it indicates that LARC inhibitors act to inhibit the cell migration toward the culture supernatant of the synovial cells.

### Example 5

### Inhibition of Migration of Peripheral Blood Mononuclear Cells by Anti-CCR6 Antibody (Measurement of the Ability of Peripheral Blood Mononuclear Cells to Migrate in Response to LARC Produced by the Synovial Cells Derived from a Rheumatoid Arthritis Patient)

The synovial cells used in Example 2 were incubated in DMEM medium containing 0.5% fetal bovine serum for 24 hours. Subsequently, the culture medium was replaced with serum-free DMEM. 10 ng/mL IL-1β and 10 ng/mL TNF-a were then added to the culture, and the culture was subsequently stimulated for 24 hours. After the culture, the culture supernatant was collected and stored at −80° C. until measurement of the chemotactic response of the peripheral blood mononuclear cells.

The peripheral blood mononuclear cells used in the measurement were prepared from 50 mL of peripheral blood collected from a healthy individual with a vacuum blood collection tube containing heparin. The assay was performed in the following manner. To the collected blood, an equal amount of PBS was added, and the mixture was carefully overlaid on top of Ficoll-Paque (available from Pharmacia) placed in a test tube. The test tube was centrifuged for 30 minutes at 1500 rpm at 20° C. to collect a layer of mononuclear cells. The mononuclear cells were suspended in DMEM containing 0.1% fetal bovine serum albumin (BSA) at a concentration of 5×10⁶ cells/mL. To the suspension, one-thousandth as much of 10 mM 5 (and 6)-carboxyfluorescein diacetate succinimidyl ester (CFSE) (available from Wako Jun-yaku) was added as a fluorescent reagent. The suspension was incubated for 20 minutes at room temperature.

The chemotactic response of the mononuclear cells was measured on a 96-well microplate (available from Neuro Probe) equipped with a polyvinylpyrrolidone-free filter with the pore size of 5 μm. The suspension of CFSE-labeled peripheral blood mononuclear cells with a cell concentration of 5×10⁶ cells/mL was added to the plate in an amount of 20 μL/well to serve as standards. The above-described supernatant of the cell culture was added to the wells in an amount of 30 μL/well to serve as a test solution. The filter was then attached to the plate, and the plate was incubated in a 5% CO₂ incubator for 15 minutes. 20 μL of the suspension of CFSE-labeled peripheral blood mononuclear cells with a cell concentration of 5×10⁶ cells/mL was added to some of the wells other than the standard wells on top of the filter. Portions of the cell suspension were independently incubated with mouse IgG2b anti-human CCR6 monoclonal antibody (30 μg/mL) (available from R&D Systems) and mouse IgG2b control antibody (30 μg/mL) (available from Pharmingen), for 30 minutes on ice. After the incubation period, 20 μL of each suspension was added to respective ones of the rest of the wells. The plate was subsequently incubated in a 5% CO₂ incubator for 4 hours and the cells remaining on the filter were removed. Filter was then removed and the fluorescence intensity was measured for each well on Cytoflour 2000 (available from Millipore).

The chemotactic responce of the peripheral blood mononuclear cells was determined by the following equation.

The chemotactic response (%)=(fluorescence intensity detected in the test well/fluorescence intensity detected in the standard well)×100

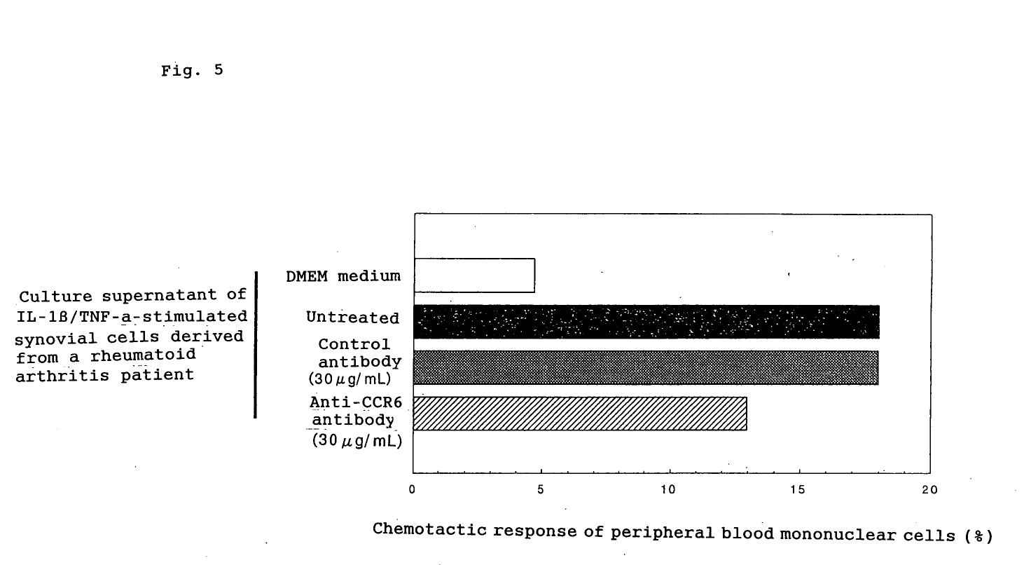

Shown in FIG. 5 are the results indicating the chemotactic response of the mononuclear cells toward LARC produced by the synovial cells. As shown, 18.0% of the mononuclear cells exhibited chemotaxis toward the culture supernatant of the synovial cells stimulated with IL-1β and TNF-a for 24 hours. In contrast, 13.0% of the mononuclear cells incubated with 30 μg/mL mouse IgG2b anti-human CCR6 monoclonal antibody, a CCR6 inhibitor, exhibited chemotaxis. This corresponds to a 5.0% decrease in the chemotactic response of the mononuclear cells as compared to the untreated mononuclear cells. Since 18.0% of the mononuclear cells incubated with mouse IgG2b control antibody exhibited chemotaxis, showing the same chemotactic response as the untreated mononuclear cells, these results suggest that anti-CCR6 antibody acts to block CCR6 on the surface of mononuclear cells and thus reduce the chemotactic response of mononuclear cells. Accordingly, it was found that CCR6 inhibitors blocked the cell migration toward the culture supernatant of the synovial cells.

### Example 6

### Expression of LARC Protein in the Synovial Tissue Derived from Rheumatoid Arthritis Patients

Synovial tissue was obtained during surgical procedures of the joint replacement of a rheumatoid arthritis patient and an osteoarthritis patient. The synovial tissue was cut into strips with scissors and was fixed in PBS containing 4% paraformaldehyde for 18 hours at 4° C. This was followed by treatment in 10% saccharose for 24 hours. The synovial tissue was embedded in OCT compound, frozen in cold acetone, and sliced into 5 μm thick using a cryostat.

The following procedures were performed at room temperature. The tissue slices were treated with methanol containing 3% hydrogen peroxide for 20 minutes and then with PBS containing 0.3% bovine serum albumin (BSA) (available from Vector Laboratories) for 20 minutes to block non-specific antibody binding. Then, these slices were blocked with PBS containing 5% goat serum for another 30 minutes. The tissue slices were then incubated with rabbit IgG anti-human LARC polyclonal antibody (20 μg/mL) (available from Pepro Tech) for 1 hour. Purified rabbit IgG antibody was used as a control for the primary antibody. After the incubation period, the tissue slices were washed three times with PBS and incubated with biotinylated goat anti-rabbit IgG polyclonal antibody (2 μg/mL)(available from Vector Laboratories, Inc) for 30 minutes. The slices were then washed three times with PBS, incubated with peroxidase-streptavidin (available from Vector Laboratories, Inc.,) for 1 hour, and again washed three times with PBS. Finally, diaminobenzidine (available from DAKO) was applied to the slices, and the slices were counterstained with hematoxylin and observed under a microscope. Shown in FIG. 6 are the results of the immunostaining performed to study expression of LARC protein in the synovial tissue. As shown, LARC protein was expressed in the synovial tissue derived from a rheumatoid arthritis patient (FIG. 6A). In comparison, as can be seen in FIG. 6B, LARC protein was not detected in the synovial tissue derived from an osteoarthritis patient. Together, these observations suggest that LARC is expressed only in the synovial tissue of rheumatoid arthritis patients, which is intensely infiltrated with deridritic cells, B-lymphocytes, and memory T-lymphocytes, but not in the synovial tissue of osteoarthritis patients, where little cell infiltration takes place. This implies that LARC is involved in the infiltration process of dendritic cells, B-lymphocytes, and memory T-lymphocytes into the synovial tissue of rheumatoid arthritis.

### Example 7

### Expression of LARC mRNA in the Synovial Tissue Derived from a Rheumatoid Arthritis Patient.

In situ hybridization was performed according to the method described by Gan et al (Gan et al., Epithelial. Cell. Biol., 1992, 1, 13). The slices of the rheumatoid synovial tissue obtained in Example 6 were treated with proteinase K (10 μg/mL) (available from Worthington Biochemical Corp.) and were placed in a hybridization solution (available from Novagen) to react with digoxigenin(DIG)-labeled sense and antisense riboprobes (50° C., 18 hrs). Subsequently, the slices were incubated with RNase (20 μg/mL) and were then reacted with anti-DIG antibody (0.75U/mL) labeled with alkaliphosphatase (available from Boehringer Mannheim)(room temperature, 60 min). Finally, 4-nitroblue tetrazolium chloride/5-bromo-4-chloro-indolyl-phosphate (NBT/BCIP) solutions (available from Roche Diagnostics GmbH) were applied to the slices, and the slices were counterstained with hematoxylin and observed under a microscope.

The sense and antisense riboprobes were prepared from the primers for LARC used in Example 1 according to the protocol described in Genetic Engineering Laboratory Manual vol. 1 & 2 (edited by Tamura Takaaki, published by Yodo-sha Co., Ltd.).

Shown in FIG. 7 are the results of in situ hybridization, indicating the expression of LARC mRNA in the synovial tissue. As can be seen, signals generated by the antisense riboprobe for LARC were detected in the synovial tissue derived from a rheumatoid arthritis patient (FIG. 7). The results indicate that LARC mRNA is expressed in the synovial tissue derived from rheumatoid arthritis patients. In comparison, LARC mRNA was not detected when the sense riboprobe for LARC was used. Collectively, these observations suggest that LARC mRNA is expressed only in the synovial tissue of rheumatoid arthritis, which is intensely infiltrated with dendritic cells, B-lymphocytes, and memory T-lymphocytes. Accordingly, it has been proven that LARC is involved in the infiltration of dendritic cells, B-lymphocytes, and memory T-lymphocytes into the synovial tissue of rheumatoid arthritis patients.

### Example 8

### Detection of CCR6-Positive Cells in the Synovial Tissue Derived from a Rheumatoid Arthritis Patient

The tissue slices of the rheumatoid synovial tissue obtained in Example 6 were treated with methanol containing 3% hydrogen peroxide for 20 minutes and then with PBS containing 0.3% bovine serum albumin (BSA) (available from Vector Laboratories) for 20 minutes to block non-specific antibody binding. Then, these slices were blocked with PBS containing 5% goat serum for anther 30 minutes. The slices were then incubated with mouse IgG 2b anti-human CCR6 monoclonal antibody (10 μg/mL) (available from R&D Systems) to serve as a primary antibody for 1 hour. Mouse IgG 2b control antibody (10 μg/mL) (available from Pharmingen) was used as a control for the primary antibody. The slices were then washed three times with PBS and incubated with biotinylated goat anti-mouse IgG polyclonal antibody (2 μg/mL) (available from DAKO) for30 minutes. The slices were then washed three times with PBS and incubated with peroxidase-streptavidin (available from Vector Laboratories, Inc.,) for 1 hour, and again washed three times with PBS. Finally, diaminobenzidine (available from DAKO) was applied to the slices, and the slices were counterstained with hematoxylin and observed under a microscope.

Shown in FIG. 8 are the results of the immunostaining performed to study the expression of CCR6 protein in the synovial tissue. As shown, CCR6 protein was expressed in the synovial tissue derived from a rheumatoid arthritis patient (FIG. 8). In comparison, no CCR6-positive cell was detected in the samples that were incubated with mouse IgG2b control antibody rather than with anti-human CCR6 antibody. Together, these observations suggest that CCR6-positive cells infiltrate into the synovial tissue of rheumatoid arthritis. This implies that LARC receptors are involved in the LARC-induced infiltration process of cells into the synovial tissue of rheumatoid arthritis.

### Example 9

### Expression of CCR6 mRNA in the Synovial Tissue Derived from a Rheumatoid Arthritis Patient

In situ hybridization was performed according to the method described by Gan et al (Gan et al., Epithelial. Cell. Biol., 1992, 1, 13). The slices of the rheumatoid synovial tissue obtained in Example 6 were treated with proteinase K (10 μg/mL) (available from Worthington Biochemical Corp.) and were placed in a hybridization solution (available from Novagen) to react with digoxigenin (DIG)-labeled sense and antisense riboprobes (50° C., 18 hrs). Subsequently, the slices were incubated with RNase (20 μg/mL) and were reacted with anti-DIG antibody (0.75U/mL) labeled with alkaliphosphatase (available from Boehringer Mannheim)(room temperature, 60 min). Finally, 4-nitroblue tetrazolium chloride/5-bromo-4-chloro-indolyl-phosphate (NBT/BCIP) solutions (available from Roche Diagnostics GmbH) were applied to the slices, and the slices were counterstained with hematoxylin and observed under a microscope. The sense and antisense riboprobes were prepared according to the protocol described in Genetic Engineering Laboratory Manual (edited by Tamura Takaaki, published by Yodo-sha Co., Ltd.) using the primers specific for CCR6: 5′-TGGATCCGTGGGGGCTGTCAGTCATCAT-3′ and 5′-TTCTCGAGCTGCCCAATAAAAGCGTAGA-3′.

Shown in FIG. 9 are the results of in situ hybridization, indicating the expression of CCR6 mRNA in the synovial tissue. As can be seen, signals generated by the antisense riboprobe for CCR6 were detected in the synovial tissue derived from a rheumatoid arthritis patient (FIG. 9). The results indicate that CCR6 mRNA is expressed in the synovial tissue derived from rheumatoid arthritis patients. In comparison, CCR6 mRNA was not detected when the sense riboprobe for CCR6 was used. Together, these observations suggest that the synovial tissues of rheumatoid arthritis patients are infiltrated with CCR6-positive cells. Accordingly, it has been proven that LARC receptors are involved in LARC-induced infiltration of cells into the synovial tissue of rheumatoid arthritis patients.

### Example 10

### Type II Collagen-Induced Arthritis (CIA) Model in Mice and Expression of LARC mRNA in the Inflamed Ankle Joints

(1) Establishing Type II Collagen-induced Arthritis Model in Mice

To induce arthritis in mice agents were prepared as follows: a solution of type II collagen (CII) derived from bovine joints (available from the Training Center for Collagen Technologies) was mixed with an equal amount of the complete Freund's adjuvant (available from Difco) to form a uniform emulsion.

Arthritis was induced in the following manner: 7-week old male DBA/1 mice were immunized with the above-prepared emulsion by intracutaneously injecting 100 μL of the emulsion (CII 150 μg) at the base of their tails. After 3 weeks, the mice were injected with the same amount of the same emulsion as a booster injection.

(2) Expression of LARC mRNA in Inflamed Ankle Joints of Mice

The time-dependency of expression of LARC mRNA in the inflamed joints was examined using real-time PCR technique. The mice immunized with CII were sacrificed every week from the first immunization to 9 weeks. The inflamed ankle joints of the sacrificed mice were stripped of skin and total RNA was extracted from the joints using ISOGEN (available from Nippon Gene). The cDNA was prepared from total RNA using the Omnitranscript RT kit (available from Qiagen). PCR was performed in a total volume of 20 μL containing 0.1 μM of each of sense primer and antisense primer for each target gene, 10 μL 2×SYBR Green PCR master mix (available from PE applied Biosystems), and 5 μL cDNA.

The primers used were: for LARC:

5′-AATCTGTGTGCGCTGATCCA-3′ and 5′-GGTTCACAGCCCTTTTCACC-3′.

Glyceraldehyde-3-phosphate dehydrogenase (GAPDH) was purchased from PE Applied Biosystems (TaqMan Rodent GAPDH Control Reagents).

PCR was performed by carrying out 40 cycles of 1) denaturation (15 sec at 95° C.), and 2) annealing and extension (1 min at 60° C.). The expression level of each target gene was quantified using Gene Amp 5700 SDS software (available from PE Applied Biosystems). Specifically, the intensity of the fluorescence signal from SYBR Green bound to the amplified PCR product (double-stranded DNA) was measured over time for each PCR cycle to draw an amplification curve of the PCR product with respect to the cycle number. The threshold cycle (Ct) was determined as the cycle at which the amplification curve intersects with a voluntary threshold value (typically a point near the midpoint of the exponential region of the amplification curve is selected).

The relative expression level of LARC mRNA with respect to GAPDH to serve as an internal standard was determined as 2^{-(Ct of LARC-Ct of GAPDH)}.

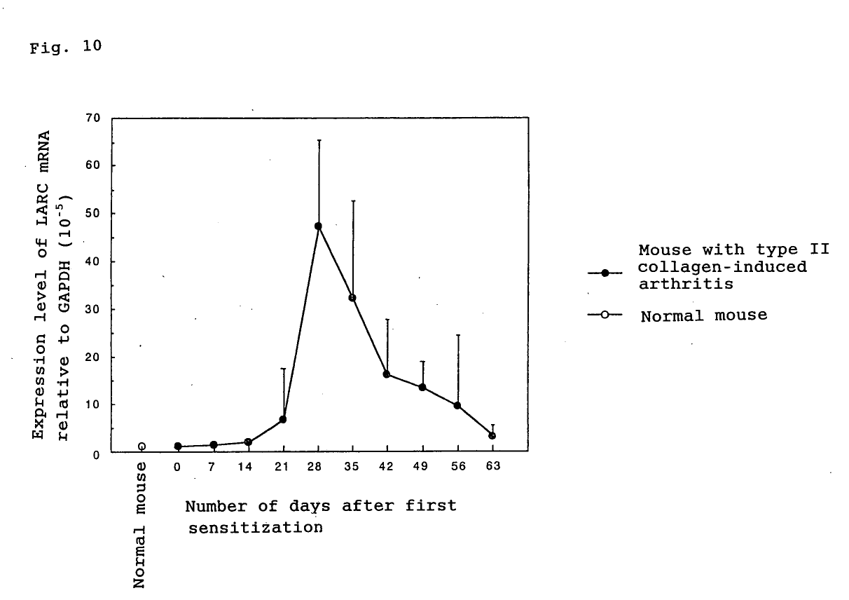

Shown in FIG. 10 are the results of the real-time PCR showing the time-dependency of expression of LARC mRNA in the inflamed joints. As can be seen, the expression level of LARC increased rapidly from day 21 to day 28 after the first immunization (peaked at day 28) and rapidly decreased until day 42. After day 42, the rate of decrease slowed down and the expression level returned to that of a normal mouse on day 63. It was shown that most of the CII-immunized mice showed the onset of the disease approximately 28 days after the first immunization and it is thought that infiltration of lymphocytes into the synovial tissue was most significant at this point. Thus, it has been shown that the expression pattern of LARC mRNA associated with the degree of symptoms of arthritis (in particular, degree of infiltration of lymphocytes into the synovium.).

### Example 11

### Expression of CCR6 mRNA in the Inflamed Ankle Joints in Mice

The time-dependency of expression of CCR6 mRNA in the inflamed joints was examined using real-time PCR technique. As in Example 10, mice immunized with CII were sacrificed every week from the first immunization to 9 weeks. The inflamed ankle joints of the sacrificed mice were stripped of skin and total RNA was extracted from the joints using ISOGEN (available from Nippon Gene). The cDNA was prepared from total RNA using the Omnitranscript RT kit (available from Qiagen). PCR was performed in a total volume of 20 μL containing 0.1 μM of each of sense primer and antisense primer for each target gene, 10 μL 2×SYBR Green PCR master mix (available from PE Applied Biosystems), and 5 μL cDNA. The used primers were: for CCR6: 5′-GGCACATATGCGGTCAACTTT-3′ and 5′-TGATACAGGCCAGGAGCAGC-3′. Glyceraldehyde-3-phosphate dehydrogenase (GAPDH) was purchased from PE Applied Biosystems (TaqMan rodent GAPDH control reagents).

PCR was performed by carrying out 40 cycles of 1) denaturation (15 sec at 95° C.), and 2) annealing and extension (1 min at 60° C.). The expression level of each target gene was quantified using Gene Amp 5700 SDS software (available from PE Applied Biosystems). Specifically, the intensity of the fluorescence signal from SYBR Green bound to the amplified PCR product (double-stranded DNA) was measured over time for each PCR cycle to draw an amplification curve of the PCR product with respect to the cycle number. The threshold cycle (Ct) was determined as the cycle at which the amplification curve intersects with a voluntary threshold value (typically a point near the midpoint of the exponential region of the amplification curve is selected).

The relative expression level of CCR6 mRNA with respect to GAPDH to serve as an internal standard was determined as 2^{-(Ct of CCR6-Ct of GAPDH)}.

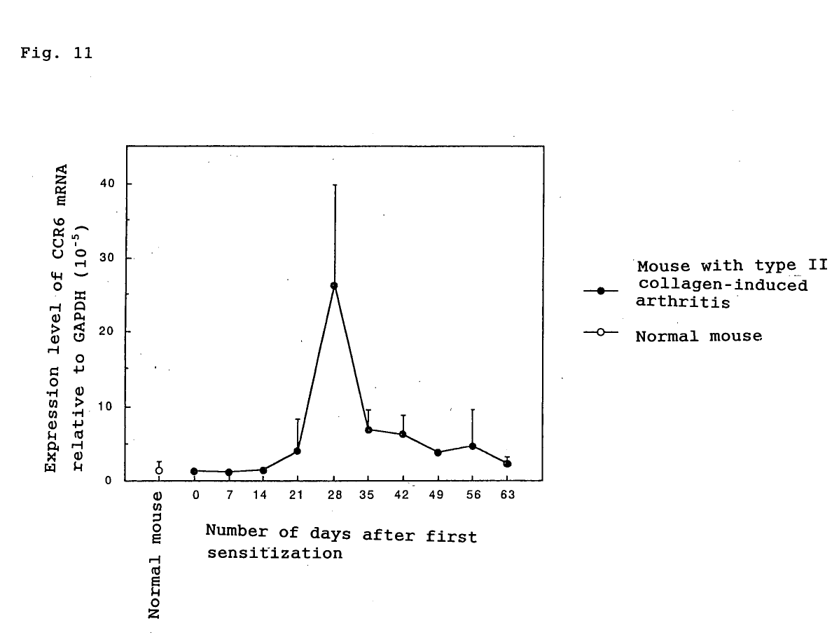

Shown in FIG. 11 are the results of real-time PCR showing the time-dependency of expression of CCR6 mRNA in the inflamed joints. As can be seen, the expression level of CCR6 increased rapidly from day 21 to day 28 after the first immunization (peaked at day 28) and rapidly decreased until day 35. After day 35, the rate of decrease slowed down and the expression level returned to that of a normal mouse on day 63. The time-dependency of expression of CCR6 mRNA substantially matched that of LARC mRNA as shown in Example 10. These observations indicate that LARC causes the cells expressing CCR6 to infiltrate into the inflamed joints in mice.

### Example 12

### Detection of Dendritic Cell Infiltrates in the Synovium of the Inflamed Ankle Joints in Mice

Ankle joints of mice in which arthritis was induced according to the method described in Example 10 (three weeks after the first immunization) was extirpated, fixed in 4% paraformaldehyde, and decalcified with 10% ethylenediaminetetraacetic acid (EDTA). The ankle joints were then embedded in paraffin and were sliced into 5 μm thick slices.

The tissue slices were then immunostained as follows: the slices were deparaffinized and were treated with methanol containing 3% hydrogen peroxide for 20 minutes and then with PBS containing 0.3% bovine serum albumin (BSA) (Vector Laboratories, Inc) for another 20 minutes to block non-specific antibody binding. The slices were then incubated with MIDC-8 antibody, a rat IgG 2a anti-mouse dendritic cell antibody (diluted 10-fold from stock solution) (available from Serotec), for 1 hour. After the incubation period, the tissue slices were washed three times with PBS and incubated with bibtinylated rabbit anti-rat Ig polyclonal antibody (diluted 500-fold from stock solution) (available from DAKO) for 30 minutes. The slices were then washed three times with PBS, incubated with peroxidase-streptavidin (available from Vector Laboratories, Inc.,) for 1 hour, and again washed three times with PBS. Finally, diaminobenzidine (available from DAKO) was applied to the slices, and the slices were counterstained with hematoxylin and observed under a microscope.

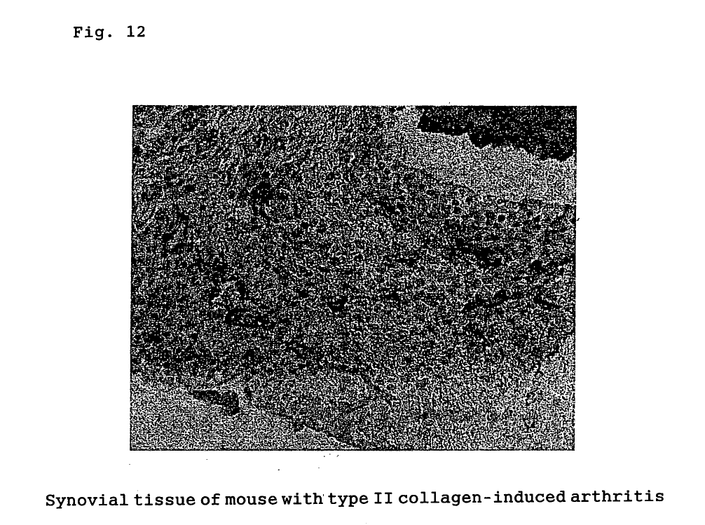

Shown in FIG. 12 are the results of the immunostaining performed to study infiltration of dendritic cells into the inflamed synovial tissues of mice. As shown, the presence of dendritic cells in the synovial tissue was detected (FIG. 12). Collectively, these observations suggest that dendritic cells infiltrate into the inflamed synovial tissues of mice.

### Example 13

### Type II Collagen-induced Arthritis (CIA) Model in Rats and Expression of LARC mRNA in the Inflamed Ankle Joints

(1) Establishing Type II Collagen-induced Arthritis Model in Rats

To induce arthritis agents were prepared as follows: a solution of type II collagen (CII) derived from bovine joints (available from the Training Center for Collagen Technologies) was mixed with the incomplete Freund's adjuvant (available from Chemicon) and N-acetylmuramyl-L-alanyl-D-isoglutamine (MDP) (available from Chemicon) to form a uniform emulsion.

Arthritis was induced in the following manner: 6-week old female Lewis rats were immunized with the above-prepared emulsion by intracutaneously injecting 1000 μL of the emulsion (CII 800 μg) in the dorsal skin. After 1 week, the rats were injected with a 100 μL (CII 80 μg) of the same emulsion as a booster immunization.

(2) Expression of LARC mRNA in the Inflamed Ankle Joints of Rats

The time-dependency of expression of LARC mRNA in the inflamed joints was examined using RT-PCR technique. The rats immunized with CII were sacrificed every week from the first immunization to 3 weeks (two normal animals and three CII-immunized animals sacrificed at a time). The inflamed joints of the sacrificed rats were stripped of skin and total RNA was extracted from the joints using ISOGEN (available from Nippon Gene). The cDNA was prepared from total RNA using the Omnitranscript RT kit (available from Qiagen). PCR was performed in a total volume of 10 μL containing 0.5 μM of each of sense primer and antisense primer for each target gene, 5 μL Hot Star Taq PCR master mix kit (available from Qiagen), and 2 μL cDNA.

The primers used were: for LARC:

5′-CCAGTCAGAAGCAGCAAGCA-3′ and 5′-CCATCCCAGAAAAGCATCCG-3′;

and for glyceraldehyde-3-phosphate dehydrogenase (GAPDH):

5′-ACCACAGTCCATGCCATCAC-3′ and 5′-TCCACCACCCTGTTGCTGTA-3′.

The conditions for the reaction were as follows: 34 cycles of 1) denaturation (20 sec at 94° C.), 2) annealing (30 sec at 60° C.), and 3) extension (30 sec at 72° C.) for amplification of the LARC gene, and 28 cycles for the GAPDH gene. A portion of the PCR products was electrophoresed on 2% agarose gel (available from Nippon Gene) containing 0.5 μg/mL ethidium,bromide (available from Sigma). Image analysis of electrophoresis was performed by Gel Doc 2000 (available from Bio-Rad).

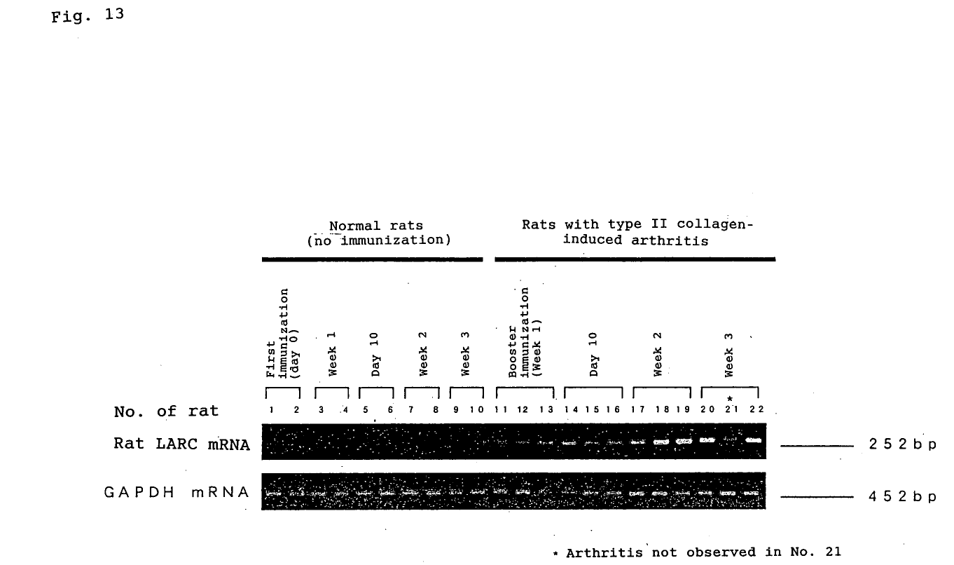

Shown in FIG. 13 are the results of the analysis on the time-dependency of expression of LARC mRNA in inflamed ankle joints of rats. As shown, LARC mRNA was strongly expressed in the arthritic rats. The expression level peaked during a period from the first immunization, when onset of disease was first observed, through week 2. In comparison, LARC mRNA was not expressed in normal rats. One rat did not experience the onset of the disease 3 weeks after the first immunization (No. 21 in FIG. 13). The expression of LARC was faint in this individual though present. Collectively, these results suggest that LARC is involved in the onset of arthritis in the model animals of rheumatoid arthritis.

### Example 14

### Expression of CCR6 mRNA in the Inflamed Ankle Joints of Rats

The time-dependency of expression of CCR6 mRNA in the inflamed joints was examined using RT-PCR technique. As in Example 13, the rats immunized with CII were sacrificed every week from the first immunization to 3 weeks (two normal animals and three CII-immunized animals sacrificed at a time). The inflamed joints of the sacrificed rats were stripped of skin and total RNA was extracted from the joints using ISOGEN (available from Nippon Gene). The cDNA was prepared from total RNA using the Omnitranscript RT kit (available from Qiagen). PCR was performed in a total volume of 10 μL containing 0.5 μM of each of sense primer and antisense primer for each target gene, 5 μL Hot Star Taq PCR master mix kit (available from Qiagen), and 2 μL cDNA.

The primers used were: for CCR6:

5′-GCTTTGTGCTCTCGTGTTAC-3′ and 5′-GGATGTGTGGTGTATGAGGA-3′;

and for glyceraldehyde-3-phosphate dehydrogenase (GAPDH):

5′-ACCACAGTCCATGCCATCAC-3′ and 5′-TCCACCACCCTGTTGCTGTA-3′.

The conditions for the reaction were as follows: 34 cycles of 1) denaturation (20 sec at 94° C.), 2) annealing (30 sec at 60° C.), and 3) extension (30 sec at 72° C.) for amplification of the LARC gene, and 28 cycles for the GAPDH gene. A portion of the PCR products was electrophoresed on 2% agarose gel (available from Nippon Gene) containing 0.5 μg/mL ethidium bromide (available from Sigma). Image analysis of electorophoresis was performed by Gel Doc 2000 (available from Bio-Rad).

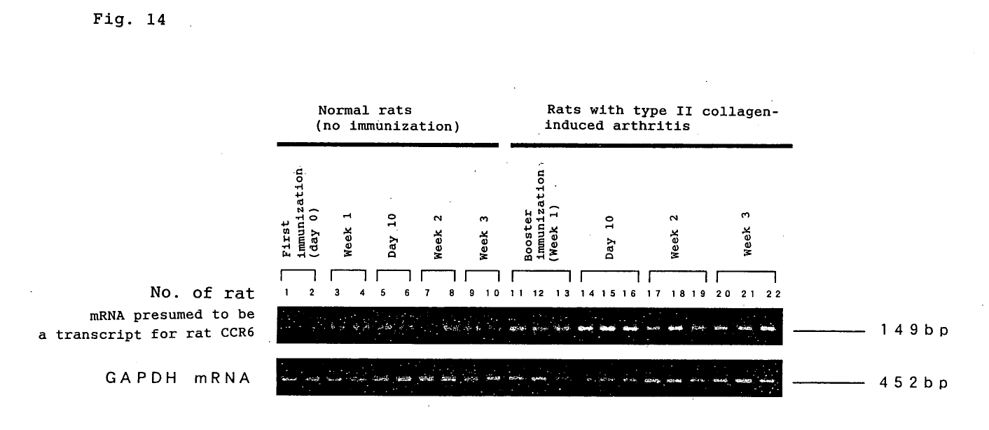

Shown in FIG. 14 are the results of the analysis on the time-dependency of mRNA that is presumed to be a transcript for CCR6 in the inflamed joints of rats. As shown, the mRNA presumed to be a transcript for CCR6 was strongly expressed in the arthritic rats. In comparison, the expression of the mRNA presumed to be a transcript for CCR6 was faint in normal rats. Collectively, these results suggest that LARC receptors are involved in the onset of arthritis in the model animals of rheumatoid arthritis.

### Example 15

### Expression of LARC Protein in the Synovium of the Inflamed Knee Joints of Rats

Knee joints of rats in which arthritis was induced according to the method described in Example 13 were extirpated, fixed in 4% paraformaldehyde, and decalcified with 10% ethylenediaminetetraacetic acid (EDTA). The knee joints were then embedded in paraffin and were sliced into 5 μm thick slices.

The tissue slices were then immunostained as follows: the slices were deparaffinized and were treated with methanol containing 3% hydrogen peroxide for 20 minutes and then with PBS containing 0.3% bovine serum albumin (BSA) (Vector Laboratories, Inc.,) for another 20 minutes to block non-specific antibody binding. The slices were then incubated with goat IgG anti-rat/mouse LARC polyclonal antibody (10 μg/mL) (available from R&D Systems) for 1 hour. Goat IgG control antibody (10 μg/mL) (available from DAKO) was used as a control for the primary antibody. After the incubation period, the tissue slices were washed three times with PBS and incubated with biotinylated rabbit anti-goat IgG polyclonal antibody (2 μg/mL) (available from DAKO) for 30 minutes. The slices were then washed three times with PBS, incubated with peroxydase-streptavidin (available from Vector Laboratories, Inc.,) for 1 hour, and again washed three times with PBS. Finally, diaminobenzidine (available from DAKO) was applied to the slices, and the slices were counterstained with hematoxylin and observed under a microscope.

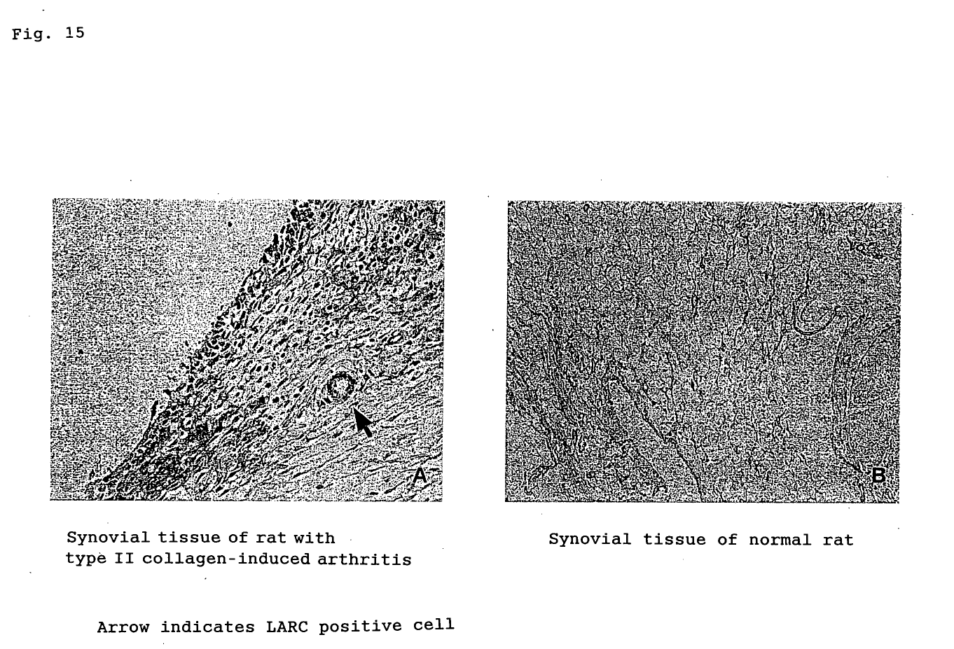

Shown in FIG. 15 are the results of the immunostaining performed to study the expression of LARC protein in the synovial tissue of the arthritic rat and a normal rat. LARC protein was detected in the endothelial cells of a blood vessel newly formed in the synovial tissue of the arthritic rat (FIG. 15a). In comparison, as shown in FIG. 15b, no positive cell was detected in the synovial tissue of the normal rat. Collectively, these observations suggest that LARC protein as well as LARC mRNA is expressed in the synovial tissue of the arthritic rats. In general, cells need to recognize chemokines in the vascular endothelial cells in order to infiltrate into the tissue. Thus, these results imply an important role of LARC protein in inducing cells to infiltrate into synovial tissue.

As it has been shown thus far, the synovial cells derived from rheumatoid arthritis patients are capable of producing LARC and thus of inducing migration of lymphocytes and peripheral blood mononuclear cells. Furthermore, the LARC inhibitor or the LARC receptor inhibitor prevented the cells from migration. Also, it has been shown that LARC is expressed in the synovial tissue of rheumatoid arthritis and of model animals of rheumatoid arthritis and that the cells expressing LARC receptors infiltrate into the synovial tissue.

Consequently, it has been suggested that inhibition of reactions mediated by LARC and by the activation of LARC receptors can lead to development of a novel treatment for rheumatoid arthritis.

1. A therapeutic or prophylactic agent for rheumatoid arthritis containing a LARC inhibitor.

2. The therapeutic or prophylactic agent for rheumatoid arthritis according to claim 1, wherein the LARC inhibitor is an antagonist of LARC.

3. The therapeutic or prophylactic agent for rheumatoid arthritis according to claim 1, wherein the LARC inhibitor is an anti-LARC antibody.

4. The therapeutic or prophylactic agent for rheumatoid arthritis according to any one of claims 1 to 3, wherein the LARC inhibitor is obtained by evaluating candidate substances using a model animal for rheumatoid arthritis.

5. A therapeutic or prophylactic agent for rheumatoid arthritis containing a LARC receptor inhibitor.

6. The therapeutic or prophylactic agent for rheumatoid arthritis according to claim 5, wherein the LARC receptor inhibitor is an antagonist of a LARC receptor.

7. The therapeutic or prophylactic agent for rheumatoid arthritis according to claim 5, wherein the LARC receptor inhibitor is an anti-LARC receptor antibody.

8. The therapeutic or prophylactic agent for rheumatoid arthritis according to any one of claims 5 to 7, wherein the LARC receptor inhibitor is obtained by evaluating candidate substances using a model animal for rheumatoid arthritis.

9. The therapeutic or prophylactic agent for rheumatoid arthritis, according to any one of claims 5 to 7, wherein the LARC receptor is CCR6.

10. A method of screening for the therapeutic or prophylactic agent for rheumatoid arthritis according to any one of claims 1 to 3 and claims 5 to 7 using an model animal for rheumatoid arthritis.

11. A method of screening for a therapeutic or prophylactic agent for rheumatoid arthritis, comprising the step of comparing the ability of a candidate substance to inhibit LARC with that of an anti-LARC antibody.

12. A method of screening for a therapeutic or prophylactic agent for rheumatoid arthritis, comprising the step of comparing the ability of a candidate substance to inhibit LARC receptor with that of an anti-LARC receptor antibody.

13. The method according to claim 12, wherein the anti-LARC receptor antibody is an anti-CCR6 antibody and the ability to inhibit the LARC receptor is the ability to inhibit CCR6.

14. A method of screening for a therapeutic or prophylactic agent for rheumatoid arthritis, comprising the step of evaluating the ability of a candidate substance to inhibit LARC-induced chemotaxis.

15. A method of screening for a therapeutic or prophylactic agent for rheumatoid arthritis, comprising the step of evaluating the ability of a candidate substance to inhibit chemotaxis induced by a supernatant of a cell culture of synovial cells derived from a rheumatoid arthritis patient.

16. A therapeutic or prophylactic agent for rheumatoid arthritis containing the substance obtained through the screening method according to any one of claims 11 to 15.

17. A therapeutic or prophylactic agent for rheumatoid arthritis containing a substance obtained by partially altering a LARC polypeptide.

18. A therapeutic or prophylactic agent for rheumatoid arthritis containing a substance obtained by genetically modifying a LARC gene.
